(19) United States
(12) Patent Application Publication
(10) Pub. No.: US 2024/0115736 A1
(43) Pub. Date: Apr. 11, 2024

(54) METHODS AND MATERIALS FOR TREATING TDP-43 PROTEINOPATHIES

(71) Applicant: Mayo Foundation for Medical Education and Research, Rochester, MN (US)

(72) Inventors: Tania Gendron, Jacksonville, FL (US); Marka Van Blitterswijk, Jacksonville Beach, FL (US)

(21) Appl. No.: 18/276,987

(22) PCT Filed: Feb. 11, 2022
(86) PCT No.: PCT/US2022/016121

§ 371 Date: Aug. 11, 2023

Related U.S. Application Data
(60) Provisional application No. 63/148,448, filed on Feb. 11, 2021.

(51) Int. Cl. A61K 48/00; A61P 25/28; C07K 14/47; C12N 15/86

(52) U.S. Cl. CPC A61K 48/005; A61P 25/28; C07K 14/4702; C12N 15/86; C12N 2750/14143

(57) ABSTRACT

Methods and materials for treating a mammal having a TDP-43 proteinopathy (a disorder characterized by the accumulation and/or aggregation of TDP-43 polypeptides in the central nervous system) are provided herein. For example, this document provides methods and materials for administering nucleic acids encoding polyadenylate-binding protein 4 (PABPC4) to a mammal having a TDP-43 proteinopathy, such that the level of PABPC4 in the central nervous system of the mammal is increased.

### CROSS-REFERENCE TO RELATED APPLICATIONS

This application claims benefit of priority from U.S. Provisional Application Ser. No. 63/148,448, filed on Feb. 11, 2021. The disclosure of the prior application is considered part of (and is incorporated by reference in) the disclosure of this application.

### STATEMENT AS TO FEDERALLY SPONSORED RESEARCH

This invention was made with government support under NS110994 awarded by the National Institutes of Health. The government has certain rights in the invention.

### TECHNICAL FIELD

This document relates to methods and materials for treating mammals having TAR DNA-binding protein 43 (TDP-43) proteinopathies, which are associated with accumulation and/or aggregation of TDP-43 in the nervous system. For example, this document provides methods and materials for administering nucleic acids encoding polyadenylate-binding protein 4 (PABPC4) to a mammal having a TDP-43 proteinopathy, such that the level of PABPC4 in the central nervous system of the mammal is increased.

### BACKGROUND

Frontotemporal dementia (FTD) and amyotrophic lateral sclerosis (ALS) are devastating neurodegenerative diseases. Patients with FTD demonstrate progressive changes in their personality and behavior, as well as language impairment (Deleon and Miller, Handb Clin Neurol 2018, 148:409-430). FTD is the second most common cause of dementia in individuals below 65 years of age (Bird et al., Ann Neurol 2003, 54(Suppl 5):S29-S31; and Harvey et al., J Neurol Neurosurg Psychiatry 2003, 74(9):1206-1209). FTD is generally divided into three groups: behavioral variant FTD (bvFTD), nonfluent-agrammatic variant primary progressive aphasia (nfvPPA), and semantic variant primary progressive aphasia (svPPA) (Pottier et al., J Neurochem 2016, 138(Suppl 1): 32-53). Although substantial clinical variability is observed with FTD, patients frequently develop symptoms between their fifth and seventh decade of life, with survival after onset usually ranging from six to eleven years (Deleon and Miller, supra). While FTD is a clinical diagnosis, the pathology associated with this disease is known as frontotemporal lobar degeneration (FTLD). FTLD has three major subgroups: FTLD-TDP, with distinctive cytoplasmic inclusions of TAR DNA-binding protein 43 (TDP-43) in the frontal cortex; FTLD-tau, with characteristic neuronal and glial inclusions of tau; and FTLD-FET, with typical inclusion bodies that contain the FUS RNA binding protein (FUS) (Pottier et al., supra).

ALS is the most common form of motor neuron disease (MND). The majority of ALS patients are in their fifties or sixties when they develop symptoms. Roughly 25% of patients present with a bulbar onset, 70% with an onset in their limbs, and 5% with a thoracic or respiratory onset (Al-Chalabi et al., Lancet Neurol 2016, 15(11):1182-1194; and Kiernan et al., Lancet 2011, 377(9769):942-955). There is no definitive diagnostic test for ALS, and the reported heterogeneity makes it challenging to diagnose. Survival after onset is relatively short, and patients often die within two to five years due to respiratory failure (Al-Chalabi et al., Nat Rev Neurol 2017, 13(2):96-104). Pathologically, ALS patients frequently exhibit cytoplasmic inclusions of TDP-43 in motor neurons.

There is considerable clinical, genetic and pathological overlap between FTD and ALS, which belong to a disease spectrum. Up to 40% of FTD patients demonstrate motor neuron involvement (Burrell et al., Brain 2011, 134(Pt 9):2582-2594; and Nguyen et al., Trends Genet 2018, 34(6):404-423), while up to 50% of ALS patients have cognitive impairment and 15% fulfill the criteria for FTD (Elamin et al., Neurology 2013, 80(17):1590-1597; and Phukan et al., J Neurol Neurosurg Psychiatry 2012, 83(1):102-108). Even within families, subjects can present with FTD, ALS or both (Kiernan et al., supra; and Morita et al., Neurology 2006, 66(6):839-844). Repeat expansions in C9orf72 are the most common known cause of both diseases (DeJesus-Hernandez et al., Neuron 2011, 72(2):245-256; and Renton et al., Neuron 2011, 72(2):257-268). TDP-43 inclusions are present in about 50% of FTD patients and more than 95% of ALS patients (Nguyen et al., supra; and Neumann et al., Science 2006, 314(5796):130-133). TDP-43 inclusions also are present in up to 63% of patients with Lewy body dementia (LBD) Robinson et al., Brain 2018, 141(7):2181-2193; McAleese et al., Brain Pathol 2017, 27(4):472-479; Bayram et al., J Alzheimer's Dis 2019, 69(4):953-961; Arai et al., Acta Neuropathol 2009, 117(2):125-136; and Nakashima-Yasuda et al., Acta Neuropathol 2007, 114(3):221-229), and in up to 56% of patients with Alzheimer's disease (Amador-Ortiz et al., Ann Neurol 2007, 61(5):435-445; Higashi et al., Brain Res 2007, 1184: 284-294; Hu et al., Acta Neuropathol 2008, 116(2):215-220; Josephs et al., Neurology 2008, 70(19 Pt 2):1850-1857; Uryu et al., J Neuropathol Exp Neurol 2008, 67(6):555-564; Arai et al., Acta Neuropathol 2009, 117(2):125-136; and Kadokura et al., Neuropathology 2009, 29(5):566-573). The cytoplasmic deposition of TDP□43 is accompanied by its loss from the nucleus, where it normally predominantly resides. In addition to being phosphorylated, pathological TDP D43 is cleaved to form C□terminal fragments (TDP-CTFs) in a region□specific manner. TDP-CTFs are enriched in the hippocampus and cortex, whereas TDP-43 lesions in spinal motor neurons are comprised primarily of full□length TDPQ43 (Igaz et al., Am J Pathol 2008, 173(1):182-194). Because of these post-translational modifications, its aggregation and its mislocalization, TDP-43 is thought to be toxic through both gain- and loss-of-function mechanisms (Gendron et al., Neuropathol Appl Neurobiol 2010, 36(2):97-112).

Despite significant advances toward unraveling pathological mechanisms underpinning FTLD-TDP, ALS, and other TDP-43 proteinopathies, no effective treatment has previously been developed for any form of these diseases.

### SUMMARY

This document provides methods and materials for treating mammals having FTLD associated with accumulation of TDP-43 polypeptides. For example, this document provides methods and materials that can be used to increase PABPC4 polypeptide levels in mammals identified as having, being likely to have, or being at increased risk of developing, TDP-43 proteinopathies. The methods provided herein can include, for example, administering a nucleic acid encoding a PABPC4 polypeptide to a mammal identified as having, or being likely to have, a TDP-43 proteinopathy.

As demonstrated herein, PABPC4 expression levels were associated with survival of patients identified as having FTLD-TDP with or without ALS. In addition, PABPC4 was demonstrated to modulate the accumulation of toxic TDP-43 products (e.g., TDP-43 fragments) in preclinical models, as overexpression of PABPC4 was associated with reduced levels of TDP-43 fragments, while suppressing expression of PABPC4 was associated with increased TDP-43 fragment levels. Having the ability to reduce the level and accumulation of TDP-43 polypeptides provides a unique and unrealized opportunity to treat mammals with disorders associated with TDP-43 pathology, such as FTD and ALS.

This document is based, at least in part, on the discovery that PABPC4 is a therapeutic target for TDP-43 proteinopathies. This document provides methods and materials for treating mammals identified as having, being likely to have, or having an increased likelihood of developing, a TDP-43 proteinopathy.

In general, one aspect of this document features methods for treating a mammal identified as having or being likely to have a TDP-43 proteinopathy. The methods can include, or consist essentially of, administering to a mammal a nucleic acid construct containing a nucleotide sequence encoding a PABPC4 polypeptide or a polyadenylate-binding protein 1 (PABPC1) polypeptide, where the administering is effective to reduce one or more symptoms of the TDP-43 proteinopathy. The nucleotide sequence can encode a PABPC4 polypeptide containing an amino acid sequence having at least 90% sequence identity to SEQ ID NO:2, SEQ ID NO:4, or SEQ ID NO:6. The PABPC4 polypeptide can contain the amino acid sequence set forth in SEQ ID NO:2, SEQ ID NO:4, or SEQ ID NO:6. The nucleotide sequence can have at least 90% sequence identity to SEQ ID NO:1, SEQ ID NO:3, or SEQ ID NO:5. The nucleotide sequence can contain the nucleotide sequence set forth in SEQ ID NO:1, SEQ ID NO:3, or SEQ ID NO:5. The nucleotide sequence can encode a PABPC1 polypeptide containing an amino acid sequence having at least 90% sequence identity to SEQ ID NO:8. The PABPC1 polypeptide can contain the amino acid sequence set forth in SEQ ID NO:8. The nucleotide sequence can have at least 90% sequence identity to SEQ ID NO:7. The nucleotide sequence can contain the nucleotide sequence set forth in SEQ ID NO:7. The nucleic acid construct can be a DNA or an RNA. The TDP-43 proteinopathy can be FTD, ALS, Alzheimer's disease, LBD, or limbic-predominant age-related TDP-43 encephalopathy (LATE). The nucleotide sequence encoding the PABPC4 polypeptide can be operably linked to a promoter. The promoter can be a non-cell-specific promoter (e.g., a cytomegalovirus (CMV) immediate-early promoter, an enhancer/chicken-0 actin promoter, a human elongation factor 1α (EF1α) promoter, a human ubiquitin C promoter, a simian virus 40 (SV40) early promoter, or a mouse phosphoglycerate kinase 1 (PGK1) promoter). The promoter can be a cell-specific promoter (e.g., a synapsin-1 promoter, an enolase promoter, a glial fibrillary acidic protein promoter, a myelin basic protein (MBP) promoter, a human myelin associated glycoprotein promoter, or an F4/80 promoter). The nucleic acid construct can be within a viral vector (e.g., an adeno-associated virus (AAV) vector, a lentivirus vector, a herpes simplex virus type 1 vector, or an adenovirus vector). The administering can include delivering the nucleic acid construct to cells in the brain of the mammal. The brain cells can be frontal cortex cells, temporal cortex cells, hippocampus cells, or motor neurons. The administering can include delivering the nucleic acid construct to cells in the spinal cord of the mammal.

In another aspect, this document features methods for reducing accumulation of a pathologic TDP-43 polypeptide within neuronal cells of a mammal identified as having, being likely to have, or being at increased risk of developing a TDP-43 proteinopathy. The methods can include, or consist essentially of, administering to a mammal a nucleic acid construct containing a nucleotide sequence encoding a PABPC4 polypeptide or a PABPC1 polypeptide. The pathologic TDP-43 polypeptide can be a TDP-43₂₀₈₋₄₁₄ fragment, a TDP-43₂₂₀₋₄₁₄ fragment, or a phosphorylated TDP-43 polypeptide. The nucleotide sequence can encode a PABPC4 polypeptide containing an amino acid sequence having at least 90% sequence identity to SEQ ID NO:2, SEQ ID NO:4, or SEQ ID NO:6. The PABPC4 polypeptide can contain the amino acid sequence set forth in SEQ ID NO:2, SEQ ID NO:4, or SEQ ID NO:6. The nucleotide sequence can have at least 90% sequence identity to SEQ ID NO:1, SEQ ID NO:3, or SEQ ID NO:5. The nucleotide sequence can contain the nucleotide sequence set forth in SEQ ID NO:1, SEQ ID NO:3, or SEQ ID NO:5. The nucleotide sequence can encode a PABPC1 polypeptide containing an amino acid sequence having at least 90% sequence identity to SEQ ID NO:8. The PABPC1 polypeptide can contain the amino acid sequence set forth in SEQ ID NO:8. The nucleotide sequence can have at least 90% sequence identity to SEQ ID NO:7. The nucleotide sequence can contain the nucleotide sequence set forth in SEQ ID NO:7. The nucleic acid construct can be a DNA or an RNA. The TDP-43 proteinopathy can be FTD, ALS, Alzheimer's disease, LBD, or LATE. The nucleotide sequence encoding the PABPC4 polypeptide can be operably linked to a promoter. The promoter can be a non-cell-specific promoter (e.g., a CMV immediate-early promoter, an enhancer/chicken-0 actin promoter, a human EF1α promoter, a human ubiquitin C promoter, a SV40 early promoter, or a mouse PGK1 promoter). The promoter can be a cell-specific promoter (e.g., a synapsin-1 promoter, an enolase promoter, a glial fibrillary acidic protein promoter, a MBP promoter, a human myelin associated glycoprotein promoter, or an F4/80 promoter). The nucleic acid construct can be within a viral vector (e.g., an AAV vector, a lentivirus vector, a herpes simplex virus type 1 vector, or an adenovirus vector). The administering can include delivering the nucleic acid construct to cells in the brain of the mammal. The brain cells can be frontal cortex cells, temporal cortex cells, hippocampus cells, or motor neurons. The administering can include delivering the nucleic acid construct to cells in the spinal cord of the mammal.

In another aspect, this document features methods for reducing one or more symptoms of a TDP-43 proteinopathy in a mammal. The methods can include, or consist essentially of, administering to a mammal a nucleic acid construct containing a nucleotide sequence encoding a PABPC4 polypeptide or PABPC1 polypeptide, where the nucleic acid construct is administered in an amount effective to reduce one or more symptoms of the TDP-43 proteinopathy in the mammal. The nucleotide sequence can encode a PABPC4 polypeptide containing an amino acid sequence having at least 90% sequence identity to SEQ ID NO:2, SEQ ID NO:4, or SEQ ID NO:6. The PABPC4 polypeptide can contain the amino acid sequence set forth in SEQ ID NO:2, SEQ ID NO:4, or SEQ ID NO:6. The nucleotide sequence can have at least 90% sequence identity to SEQ ID NO:1, SEQ ID NO:3, or SEQ ID NO:5. The nucleotide sequence can contain the nucleotide sequence set forth in SEQ ID NO:1, SEQ ID NO:3, or SEQ ID NO:5. The nucleotide sequence can encode a PABPC1 polypeptide containing an amino acid sequence having at least 90% sequence identity to SEQ ID NO: 8. The PABPC1 polypeptide can contain the amino acid sequence set forth in SEQ ID NO:8. The nucleotide sequence can have at least 90% sequence identity to SEQ ID NO:7. The nucleotide sequence can contain the nucleotide sequence set forth in SEQ ID NO:7. The nucleic acid construct can be a DNA or an RNA. The TDP-43 proteinopathy can be FTD, ALS, Alzheimer's disease, LBD, or LATE. The nucleotide sequence encoding the PABPC4 polypeptide can be operably linked to a promoter. The promoter can be a non-cell-specific promoter (e.g., a CMV immediate-early promoter, an enhancer/chicken-0 actin promoter, a human EF1α promoter, a human ubiquitin C promoter, a SV40 early promoter, or a mouse PGK1 promoter). The promoter can be a cell-specific promoter (e.g., a synapsin-1 promoter, an enolase promoter, a glial fibrillary acidic protein promoter, a MBP promoter, a human myelin associated glycoprotein promoter, or an F4/80 promoter). The nucleic acid construct can be within a viral vector (e.g., an AAV vector, a lentivirus vector, a herpes simplex virus type 1 vector, or an adenovirus vector). The administering can include delivering the nucleic acid construct to cells in the brain of the mammal. The brain cells can be frontal cortex cells, temporal cortex cells, hippocampus cells, or motor neurons. The administering can include delivering the nucleic acid construct to cells in the spinal cord of the mammal.

In another aspect, this document features methods for treating a mammal identified being at increased likelihood of developing a TDP-43 proteinopathy. The methods can include, or consist essentially of, administering to a mammal a nucleic acid construct containing a nucleotide sequence encoding a PABPC4 polypeptide or a PABPC1 polypeptide, wherein the administering is effective to delay or prevent the onset of one or more symptoms of the TDP-43 proteinopathy. The mammal can be identified as being at increased likelihood of developing the TDP-43 proteinopathy based on detection of a C9orf72 mutation, a GRN mutation, a VCP mutation, a TARDBP mutation, an HNRNPA2B1 mutation, a SETX mutation, a DCTN1 mutation, an ATXN2 mutation, a UNCI3A mutation, a DPP6 mutation, a TMEM106B mutation, an ANG mutation, and/or NIPA1 mutation in the mammal. The nucleotide sequence can encode a PABPC4 polypeptide containing an amino acid sequence having at least 90% sequence identity to SEQ ID NO:2, SEQ ID NO:4, or SEQ ID NO:6. The PABPC4 polypeptide can contain the amino acid sequence set forth in SEQ ID NO:2, SEQ ID NO:4, or SEQ ID NO:6. The nucleotide sequence can have at least 90% sequence identity to SEQ ID NO:1, SEQ ID NO:3, or SEQ ID NO:5. The nucleotide sequence can contain the nucleotide sequence set forth in SEQ ID NO:1, SEQ ID NO:3, or SEQ ID NO:5. The nucleotide sequence can encode a PABPC1 polypeptide containing an amino acid sequence having at least 90% sequence identity to SEQ ID NO:8. The PABPC1 polypeptide can contain the amino acid sequence set forth in SEQ ID NO:8. The nucleotide sequence can have at least 90% sequence identity to SEQ ID NO:7. The nucleotide sequence can contain the nucleotide sequence set forth in SEQ ID NO:7. The nucleic acid construct can be a DNA or an RNA. The TDP-43 proteinopathy can be FTD, ALS, Alzheimer's disease, LBD, or LATE. The nucleotide sequence encoding the PABPC4 polypeptide can be operably linked to a promoter. The promoter can be a non-cell-specific promoter (e.g., a CMV immediate-early promoter, an enhancer/chicken-0 actin promoter, a human EF1α promoter, a human ubiquitin C promoter, a SV40 early promoter, or a mouse PGK1 promoter). The promoter can be a cell-specific promoter (e.g., a synapsin-1 promoter, an enolase promoter, a glial fibrillary acidic protein promoter, a MBP promoter, a human myelin associated glycoprotein promoter, or an F4/80 promoter). The nucleic acid construct can be within a viral vector (e.g., an AAV vector, a lentivirus vector, a herpes simplex virus type 1 vector, or an adenovirus vector). The administering can include delivering the nucleic acid construct to cells in the brain of the mammal. The brain cells can be frontal cortex cells, temporal cortex cells, hippocampus cells, or motor neurons. The administering can include delivering the nucleic acid construct to cells in the spinal cord of the mammal.

Unless otherwise defined, all technical and scientific terms used herein have the same meaning as commonly understood by one of ordinary skill in the art to which this invention pertains. Although methods and materials similar or equivalent to those described herein can be used to practice the invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

The details of one or more embodiments of the invention are set forth in the accompanying drawings and the description below. Other features, objects, and advantages of the invention will be apparent from the description and drawings, and from the claims.

### DESCRIPTION OF THE DRAWINGS

FIGS. 1A-1G show a graphical representation of PABPC4 transcript variants and protein domains, and representative nucleic acid and amino acid sequences for human PABPC4 isoforms. FIG. 1A is a diagram of three RefSeq transcript variants. Variant 1 (SEQ ID NO:1; FIG. 1B) encodes a 660-amino-acid protein (SEQ ID NO:2; FIG. 1C). Variant 2 (SEQ ID NO:3; FIG. 1D) encodes a 644-amino-acid protein (SEQ ID NO:4; FIG. 1E). Variant 3 (SEQ ID NO:5; shown in FIG. 1F) encodes a 631-amino-acid protein (SEQ ID NO:6; FIG. 1G). The full length PABPC4 polypeptides contain 4 RNA recognition motifs (RRMs) and one domain that is characteristic for the PABP family of proteins.

FIG. 2A shows a representative nucleotide sequence for a human PABPC1 polypeptide (SEQ ID NO:7), and FIG. 2B shows a representative amino acid sequence for human PABPC1 polypeptide (SEQ ID NO:8).

FIGS. 3A-3F are Kaplan-Meier curves showing that PABPC4 RNA expression was associated with survival after disease onset (comparing the bottom 50% to the top 50% of RNA expression levels). Higher expression levels of PABPC4 were associated with prolonged survival, either with (FIG. 3A) or without (FIG. 3B) adjustment for cellular composition. When taking cellular composition into consideration, similar patterns were obtained when restricting the analysis to FTLD-TDP patients (FIG. 3C) or to FTLD/ALS patients (FIG. 3D) as well as to C9orf72 expansion carriers (c9; FIG. 3E) or non-expansion carriers (non-c9; FIG. 3F).

FIG. 4 includes images of Western blots showing that PABPC4 modulated TDP-CTF accumulation. Left panel: Myc-tagged PABPC4 (Myc-PABPC4) or Myc alone were overexpressed in cultured HEK293T cells expressing GFP-TDP₂₂₀₋₄₁₄, and soluble (Sol) and insoluble (Insol) protein lysates were evaluated by Western blot using antibodies against GFP or phosphorylated TDP-43. Overexpressing PABPC4 attenuated the accumulation of GFP-TDP₂₂₀₋₄₁₄. Right panel: HEK293T cells were treated with a control siRNA (siCTL) or with an siRNA towards PABPC4 (siPABPC4) to knock down PABPC4. Knocking down PABPC4 augmented the accumulation of insoluble GFP-TDP₂₂₀₋₄₁₄.

FIG. 5 includes images of Western blots showing that PABPC4 attenuated insoluble TDP-CTF accumulation in M17 (neuroblastoma) cells, while decreasing PABPC4 increased insoluble TDP-CTF accumulation. PABPC4 was overexpressed (Myc-PABPC4) or knocked-down (siPABPC4) in cultured M17 cells expressing GFP-TDP₂₂₀₋₄₁₄. Blots using insoluble protein lysates are shown. GFP-TDP₂₂₀₋₄₁₄ was examined using antibodies against total or phosphorylated TDP-43. HMW: high molecular weight GFP-TDP₂₂₀₋₄₁₄ oligomers.

FIGS. 6A and 6B include images of Western blots showing that PABPC4 modulated the accumulation of phosphorylated TDP-CTF and cytoplasmic full-length TDP-43. FIG. 6A: PABPC4 was overexpressed in cultured HEK293T cells expressing the TDP-43 fragment GFP-TDP₂₀₈₋₄₁₄ or expressing GFP-TDP-43_{NLSmut}, which localizes predominantly to the cytoplasm due to the introduction of mutations in the TDP-43 nuclear localization signal. Overexpression of PABPC4 attenuated levels of phosphorylated GFP-TDP₂₀₈₋₄₁₄ and GFP-TDP-43_{NLSmut}. FIG. 6B: PABPC4 was knocked-down in cultured HEK293T cells expressing the TDP-43 fragment GFP-TDP₂₀₈₋₄₁₄ or expressing GFP-TDP-43_{NLSmut}. Depletion of PABPC4 increased phosphorylated GFP-TDP₂₀₈₋₄₁₄ and GFP-TDP-43_{NLSmut}. Protein lysates were evaluated by Western blot. GFP-TDP₂₀₈₋₄₁₄ and GFP-TDP-43_{NLSmut} were examined using antibodies against total TDP-43 and phosphorylated TDP-43.

FIG. 7 includes images of cells co-expressing GFP-TDP-43_{NLSmut} and either myc-PABPC4 or myc alone immunostained with an anti-PABPC4 antibody and a fluorescently tagged secondary antibody. Compared to GFP-positive cells with only endogenous PABPC4, GFP-positive cells with PABPC4 overexpression had fewer GFP-TDP-43_{NLSmut} aggregates, with GFP-TDP-43_{NLSmut} being present in a more diffuse fashion. Endogenous PABPC4 is not visible in these images since all images were taken with the same exposure time, which was very short for cells expressing myc-PABPC4. The pie charts at the right of the figure plot the percentages of aggregated TDP-43, diffuse TDP-43, and aggregated and diffuse TDP-43.

FIG. 8 includes images of Western blots showing that PABPC1 attenuated accumulation of phosphorylated TDP-CTF in HEK293T cells. PABPC1 was overexpressed in cultured HEK293T cells expressing GFP-TDP₂₀₈₋₄₁₄, and protein lysates were evaluated by Western blot. GFP-TDP₂₀₈₋₄₁₄ was examined using antibodies against total TDP-43 and phosphorylated TDP-43.

### DETAILED DESCRIPTION

PABPC4 is a member of the cytoplasmic poly(A)-binding protein (PABPC) family of polypeptides. PABPC4 binds mRNA 3′ poly(A) tails, and plays an important role in mRNA stability and translation initiation. PABPC4, and the related PABPC1 polypeptide, are predominantly cytoplasmic, although they shuttle between the cytoplasm and nucleus (Afonina et al., J Biol Chem 1998, 273(21):13015-13021; and Burgess et al., J Cell Sci 2011, 124(Pt 19):3344-3355). PABPC4 and PABPC1 can interact with TDP-43 (Freibaum et al., J Proteome Res 2010, 9(2):1104-1120; Dammer et al., PLoS One 2012, 7(6):e38658; Ling et al., Proc Natl Acad Sci USA 2010, 107(30):13318-13323; and Blokhuis et al., Acta Neuropathol 2016, 132(2):175-196). Like TDP-43, both are components of stress granules—membraneless organelles that temporarily assemble upon cellular insults to preserve cell viability (Kuechler et al., J Mol Biol 2020, 432(7):2349-2368).

This document provides methods and materials for treating mammals identified as having, being likely to have, or being at increased risk of developing, a TDP-43 proteinopathy by increasing the level of PABPC4 or a related polypeptide (e.g., PABPC1) in the mammals. In general, the methods and materials provided herein involve the use of nucleic acid constructs that contain a nucleic acid encoding a PABPC (e.g., PABPC4) polypeptide. The methods and materials provided herein can be used to reduce one or more symptoms of the TDP-43 proteinopathy, and/or to reduce the amount of an aggregated TDP-43 polypeptide in cells (e.g., neural cells) of the mammals being treated.

In some cases, this document provides nucleic acids that can be used to treat a mammal having a TDP-43 proteinopathy or, in some cases, another disorder associated with improper protein aggregation. Disorders that can be treated using the methods provided herein include, without limitation, FTD, ALS, Alzheimer's disease, LBD, limbic-predominant age-related TDP-43 encephalopathy (LATE; Nelson et al., Brain 2019, 142(6):1503-1527; erratum in: Brain 2019, 142(7):e37), and other conditions associated with the accumulation of pathological TDP-43.

The term “nucleic acid” as used herein encompasses both RNA (e.g., mRNA) and DNA, including cDNA, genomic DNA, and synthetic (e.g., chemically synthesized) DNA. A nucleic acid can be double-stranded or single-stranded. A single-stranded nucleic acid can be the sense strand or the antisense strand. In addition, a nucleic acid can be circular or linear. The term “isolated,” when in reference to a nucleic acid, refers to a nucleic acid that is separated from other nucleic acids that are present in a genome, e.g., a mammalian genome, including nucleic acids that normally flank one or both sides of the nucleic acid in the genome. The term “isolated” as used herein with respect to nucleic acids also includes any non-naturally-occurring sequence, since such non-naturally-occurring sequences are not found in nature and do not have immediately contiguous sequences in a naturally-occurring genome.

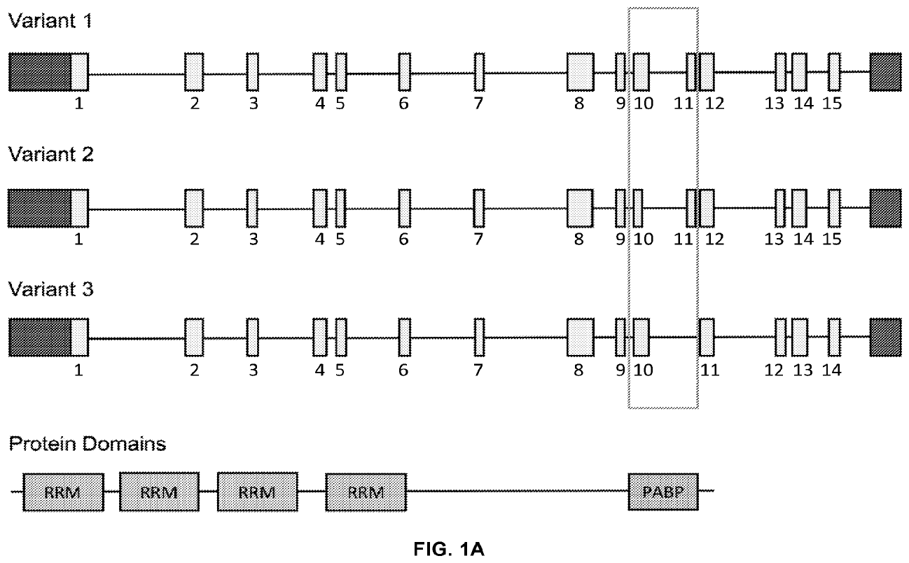

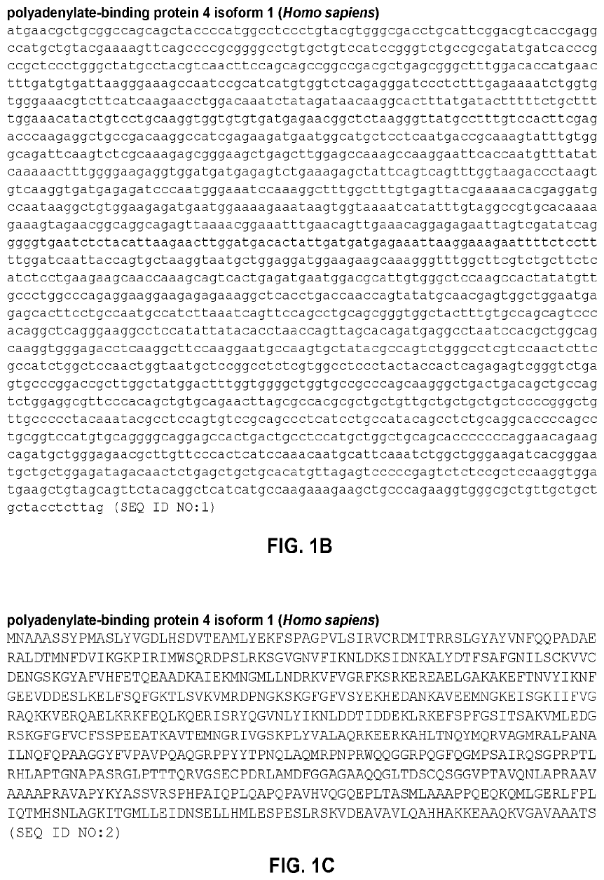

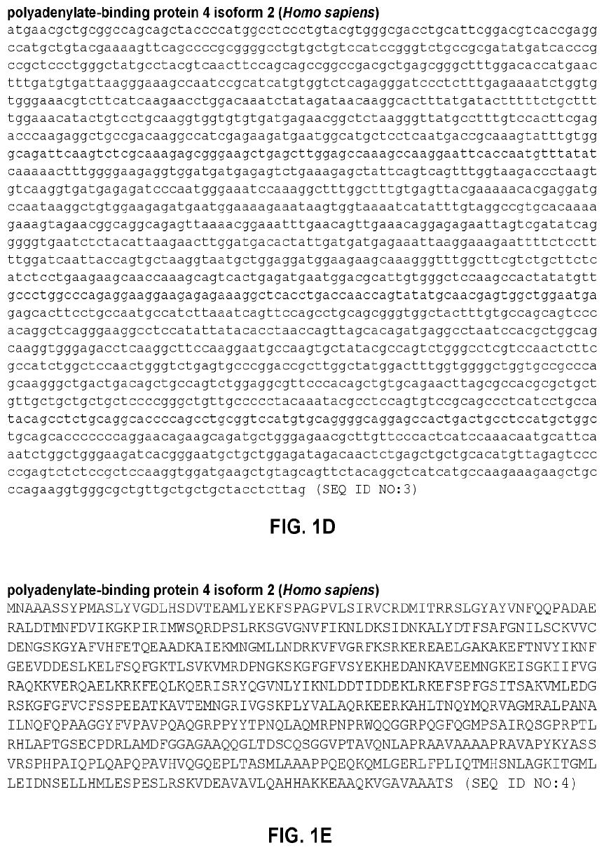

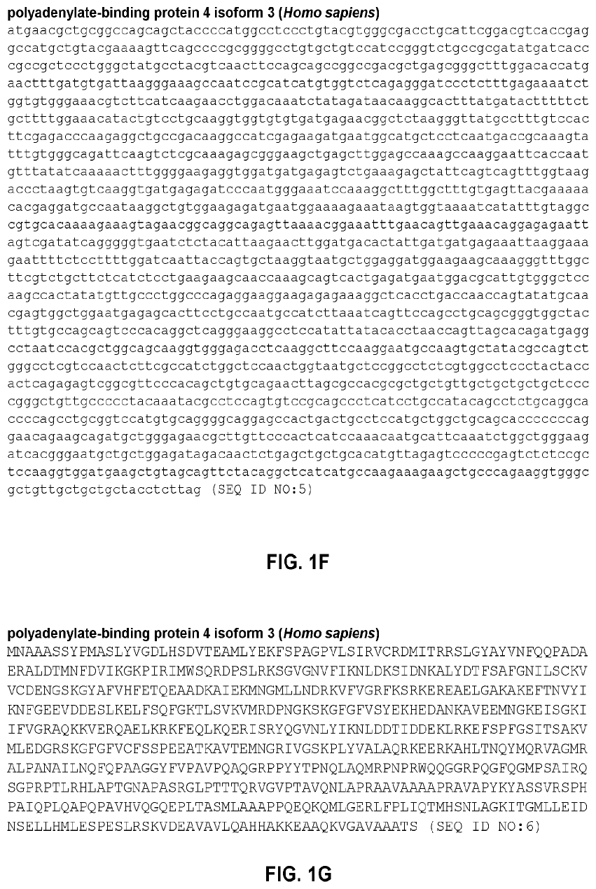

The nucleic acids provided herein include a nucleotide sequence encoding a PABPC polypeptide (e.g., a PABPC4 polypeptide or a PABPC1 polypeptide). In some cases, a PABPC polypeptide can be a PABPC4 polypeptide. PABPC4 has several transcript variants (FIG. 1A) that encode several polypeptide variants. In some cases, a PABPC4 polypeptide can be a PABPC4 isoform 1 polypeptide encoded by the sequence set forth in SEQ ID NO:1 (FIG. 1B) and having the amino acid sequence set forth in SEQ ID NO:2 (FIG. 1C). In some cases, a PABPC4 polypeptide can be a PABPC4 isoform 2 polypeptide encoded by the nucleotide sequence set forth in SEQ ID NO:3 (FIG. 1D) and having the amino acid sequence set forth in SEQ ID NO:4 (FIG. 1E). In some cases, a PABPC4 polypeptide can be a PABPC4 isoform 3 polypeptide encoded by the nucleotide sequence set forth in SEQ ID NO:5 (FIG. 1F) and having the amino acid sequence set forth in SEQ ID NO:6 (FIG. 1G). Sequences for PABPC4 nucleic acids and polypeptides are available in GENBANK®. For example, a PABPC4 variant 1 nucleotide (mRNA) sequence is available under NCBI ref. NM_001135653 (e.g., version NM_001135653.2), and a PABPC4 isoform 1 amino acid sequence is available under NCBI ref. NP_001129125 (e.g., version NP_001129125.1). A PABPC4 variant 2 nucleotide (mRNA) sequence is available under NCBI ref. NM_003819 (e.g., version NM_003819.4), and a PABPC4 isoform 2 amino acid sequence is available under NCBI ref. NP_003810 (e.g., version NP_003810.1). A PABPC4 variant 3 nucleotide (mRNA) sequence is available under NCBI ref. NM_001135654 (e.g., version NM_001135654.2), and a PABPC4 isoform 3 amino acid sequence is available under NCBI ref. NP_001129126 (e.g., version NP_001129126.1).

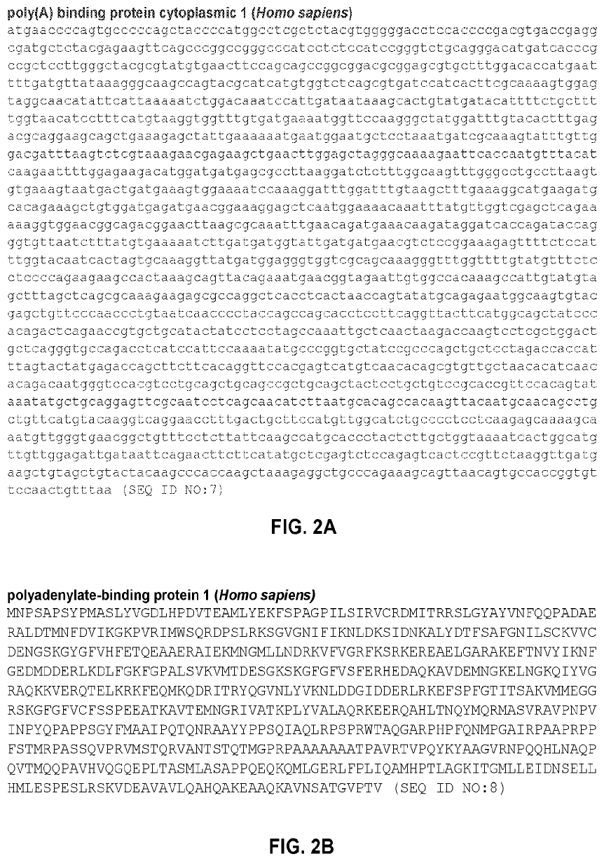

In some cases, a PABPC polypeptide can be a PABPC1 polypeptide. For example, a PABPC1 polypeptide can be encoded by the nucleotide sequence set forth in SEQ ID NO:7 (FIG. 2A), and can have the amino acid sequence set forth in SEQ ID NO:8 (FIG. 2B). Sequences for PABPC1 nucleic acids and polypeptides are available in GENBANK®. For example, a PABPC1 nucleotide (mRNA) sequence is available under NCBI ref. NM_002568 (e.g., version NM_002568.4), and a PABPC1 amino acid sequence is available under NCBI ref. NCBI ref. NP_002559 (e.g., version NCBI ref. NP_002559.2).

In some cases, a nucleotide sequence encoding a PABPC polypeptide (e.g., a PABPC4 polypeptide or a PABPC1 polypeptide) can be at least 90 percent (e.g., at least 91, 92, 93, 94, 95, 96, 97, 98, or 99 percent) identical to the nucleotide sequence set forth in SEQ ID NO:1, SEQ ID NO:3, SEQ ID NO:5, or SEQ ID NO:7. In some cases, a PABPC polypeptide can have an amino acid sequence that is at least 90 percent (e.g., at least 91, 92, 93, 94, 95, 96, 97, 98, or 99 percent) identical to the sequence set forth in SEQ ID NO:2, SEQ ID NO:4, SEQ ID NO:6, or SEQ ID NO:8.

The percent sequence identity between a particular nucleic acid or amino acid sequence and a sequence referenced by a particular sequence identification number is determined as follows. First, a nucleic acid or amino acid sequence is compared to the sequence set forth in a particular sequence identification number using the BLAST 2 Sequences (B12seq) program from the stand-alone version of BLASTZ containing BLASTN version 2.0.14 and BLASTP version 2.0.14. This stand-alone version of BLASTZ can be obtained online at fr.com/blast or at ncbi.nlm.nih.gov. Instructions explaining how to use the B12seq program can be found in the readme file accompanying BLASTZ. B12seq performs a comparison between two sequences using either the BLASTN or BLASTP algorithm.

BLASTN is used to compare nucleic acid sequences, while BLASTP is used to compare amino acid sequences. To compare two nucleic acid sequences, the options are set as follows: -i is set to a file containing the first nucleic acid sequence to be compared (e.g., C:\seq1.txt); -j is set to a file containing the second nucleic acid sequence to be compared (e.g., C:\seq2.txt); -p is set to blastn; -o is set to any desired file name (e.g., C:\output.txt); -q is set to -1; -r is set to 2; and all other options are left at their default setting. For example, the following command can be used to generate an output file containing a comparison between two sequences: C:\B12seq-i c:\seq1.txt-j c:\seq2.txt-p blastn-o c:\output.txt-q-1-r 2. To compare two amino acid sequences, the options of B12seq are set as follows: -i is set to a file containing the first amino acid sequence to be compared (e.g., C:\seq1.txt); -j is set to a file containing the second amino acid sequence to be compared (e.g., C:\seq2.txt); -p is set to blastp; -o is set to any desired file name (e.g., C:\output.txt); and all other options are left at their default setting. For example, the following command can be used to generate an output file containing a comparison between two amino acid sequences: C:\B12seq-i c:\seq1.txt-j c:\seq2.txt-p blastp-o c:\output.txt. If the two compared sequences share homology, then the designated output file will present those regions of homology as aligned sequences. If the two compared sequences do not share homology, then the designated output file will not present aligned sequences.

PABPC polypeptides (e.g., PABPC4 or PABPC1 polypeptides) that are not 100% identical to SEQ ID NO:2, SEQ ID NO:4, SEQ ID NO:6, or SEQ ID NO:8 can include one or more (e.g., one, two, three, four, five, six, seven, eight, nine, ten, or more than ten) amino acid substitutions, additions, or subtractions as compare to SEQ ID NO:2, SEQ ID NO:4, SEQ ID NO:6, or SEQ ID NO:8. Amino acid substitutions can be made, in some cases, by selecting substitutions that do not differ significantly in their effect on maintaining (a) the structure of the peptide backbone in the area of the substitution, (b) the charge or hydrophobicity of the molecule at particular sites, or (c) the bulk of the side chain. For example, naturally occurring residues can be divided into groups based on side-chain properties: (1) hydrophobic amino acids (methionine, alanine, valine, leucine, and isoleucine); (2) neutral hydrophilic amino acids (cysteine, serine, and threonine); (3) acidic amino acids (aspartic acid and glutamic acid); (4) basic amino acids (asparagine, glutamine, histidine, lysine, and arginine); (5) amino acids that influence chain orientation (glycine and proline); and (6) aromatic amino acids (tryptophan, tyrosine, and phenylalanine). Substitutions made within these groups can be considered conservative substitutions. Non-limiting examples of conservative substitutions that can be encoded by a PABPC-encoding nucleic acid provided herein include, without limitation, substitution of valine for alanine, lysine for arginine, glutamine for asparagine, glutamic acid for aspartic acid, serine for cysteine, asparagine for glutamine, aspartic acid for glutamic acid, proline for glycine, arginine for histidine, leucine for isoleucine, isoleucine for leucine, arginine for lysine, leucine for methionine, leucine for phenylalanine, glycine for proline, threonine for serine, serine for threonine, tyrosine for tryptophan, phenylalanine for tyrosine, and/or leucine for valine.

Nucleic acid molecules encoding a PABPC polypeptide (e.g., a PABPC4 polypeptide or a PABPC1 polypeptide) can be produced by techniques including, without limitation, common molecular cloning, polymerase chain reaction (PCR), chemical nucleic acid synthesis techniques, and combinations of such techniques. For example, PCR can be used with oligonucleotide primers designed to amplify nucleic acid (e.g., genomic DNA or RNA) encoding a selected polypeptide (e.g., a PABPC4 polypeptide or a PABPC1 polypeptide).

In some cases, a nucleic acid encoding a PABPC polypeptide (e.g., a PABPC4 polypeptide or a PABPC1 polypeptide) can be included in a recombinant nucleic acid construct (e.g., a vector). A “vector” is a replicon, such as a plasmid, phage, or cosmid, into which another DNA segment may be inserted so as to bring about the replication of the inserted segment. Generally, a vector is capable of replication when associated with the proper control elements. Any appropriate vector backbone can be used, including, for example, plasmids or viruses. The term “vector” includes cloning and expression vectors, as well as viral vectors and integrating vectors. An “expression vector” is a vector that includes one or more expression control sequences, and an “expression control sequence” is a DNA sequence that controls and regulates the transcription and/or translation of another DNA sequence. Expression vectors include, without limitation, plasmids and viral vectors derived from, for example, bacteriophage, baculoviruses, tobacco mosaic virus, herpes viruses, cytomegalovirus, retroviruses, vaccinia viruses, adenoviruses, and adeno-associated viruses.

In some cases, a nucleotide sequence encoding a PABPC polypeptide (e.g., a PABPC4 polypeptide or a PABPC1 polypeptide) can be operably to one or more regulatory regions. The term “regulatory region” (sometimes referred to as an “expression control sequence” or “control element”) refers to nucleotide sequences that influence transcription or translation initiation and rate, and stability and/or mobility of a transcript or polypeptide product. Regulatory regions can include, without limitation, promoter sequences, enhancer sequences, response elements, protein recognition sites, inducible elements, promoter control elements, protein binding sequences, 5′ and 3′ untranslated regions (UTRs), transcriptional start sites, termination sequences, polyadenylation sequences, introns, and other regulatory regions that can reside within coding sequences, such as secretory signals, Nuclear Localization Sequences (NLS) and protease cleavage sites.

As used herein, “operably linked” means that a regulatory region and a coding sequence are incorporated into a construct so that expression of the regulator region effectively controls expression of the coding sequence. A coding sequence is “operably linked” to an expression control sequence in a cell when RNA polymerase is able to transcribe the coding sequence into RNA, which if an mRNA, then can be translated into the protein encoded by the coding sequence. Thus, a regulatory region can modulate, e.g., regulate, facilitate or drive, transcription in the cells, tissue, organ, or mammal in which it is desired to express a polypeptide.

In some cases, a nucleotide sequence encoding a PABPC polypeptide (e.g., a PABPC4 polypeptide or a PABPC1 polypeptide) can be operably linked to a promoter that can control when and where the polypeptide is expressed. The choice of promoters to be included depends upon factors including, without limitation, efficiency, selectability, inducibility, desired expression level, and cell or tissue specificity. The promoter can be a constitutive promoter, an inducible promoter, or a cell-type specific promoter. For example, tissue-, organ- and cell-specific promoters that confer transcription only or predominantly in a particular tissue, organ, and cell type, respectively, can be used. Inducible promoters can confer transcription in response to external stimuli such as chemical agents, developmental stimuli, or environmental stimuli.

Any appropriate promoter can be operably linked to a nucleotide sequence encoding a PABPC polypeptide (e.g., a PABPC4 polypeptide or a PABPC1 polypeptide) in the nucleic acid constructs provided herein. Examples of promoters that can drive expression in a non-cell specific manner and can be used in the nucleic acid constructs provided herein include, without limitation, the CMV immediate-early promoter, the enhancer/chicken-0 actin promoter, the human EF1α promoter, the human ubiquitin C promoter, the SV40 early promoter, and the mouse PGK1 promoter. Examples of promoters that can drive cell-specific expression and can be used in the nucleic acid constructs provided herein include, without limitation, the synapsin-1 promoter or the neuron-specific enolase promoter for neuron-specific expression, the glial fibrillary acidic protein promoter for astrocyte-specific expression, the MBP promoter or the human myelin associated glycoprotein promoter for oligodendrocyte-specific expression, and the F4/80 promoter for microglia-specific expression.

In some cases, a nucleic acid construct provided herein can include a 5′ UTR, a 3′ UTRs, and/or a polyadenylation signal. A 5′ UTR is transcribed but not translated, lies between the start site of the transcript and the translation initiation codon, and may include the +1 nucleotide. A 3′ UTR can be positioned between the translation termination codon and the end of the transcript. UTRs can have particular functions, such as increasing mRNA message stability or translation attenuation. An example of a 3′ UTR a polyadenylation signal.

In some cases, a nucleic acid containing a sequence encoding a PABPC polypeptide (e.g., a PABPC4 polypeptide or a PABPC1 polypeptide) can be contained in a viral vector. Any appropriate viral vector can be used. Examples of suitable viral vectors include, without limitation, parvovirus (e.g., adeno-associated virus), lentivirus, herpes simplex virus type 1, and adenovirus.

A vector can be “non-integrative” or “integrative.” A non-integrative vector is a vector that does not integrate the genome of a cell. Non-integrative vectors can be capable of autonomous, extra-chromosomal replication and/or expression of nucleic acids contained within the vector sequences. An integrative vector can integrate into the genome of a cell (e.g., through the action of a virus integrase, or through homologous recombination). In some cases, for example, a recombinant nucleic acid provided herein can integrate into the genome of a cell via illegitimate (random, non-homologous, non site-specific) recombination. In some cases, a recombinant nucleic acid provided herein can be adapted to integrate into the genome of a cell via homologous recombination. For example, nucleic acid sequences adapted for integration via homologous recombination can be flanked on both sides with sequences that are similar or identical to endogenous target nucleotide sequences, which can facilitate integration of the recombinant nucleic acid at a particular site in the genome containing the endogenous target nucleotide sequences. In some cases, a recombinant nucleic acid sequence can be adapted to integrate into the genome of a cell via site-specific recombination that occurs when a nucleic acid sequence is targeted to a particular site within a genome not by homology between sequences in the recombinant nucleic acid and sequences in the genome, but rather by the action of recombinase enzymes that recognize specific nucleic acid sequences and catalyze the reciprocal exchange of DNA strands between these sites. Site-specific recombination thus includes enzyme-mediated cleavage and ligation of two defined nucleotide sequences. Site-specific recombination systems include, for example, the Cre-lox system and the FLP-FRT system.

In some cases, a nucleic acid containing a nucleotide sequence encoding a PABPC polypeptide (e.g., a PABPC4 polypeptide or a PABPC1 polypeptide) can be formulated into a pharmaceutically acceptable composition. For example, a nucleic acid provided herein can be formulated together with one or more pharmaceutically acceptable carriers (additives) and/or diluents. Pharmaceutically acceptable carriers, diluents, adjuvants, and vehicles that can be used in the pharmaceutical compositions provided herein include, without limitation, sterile water, saline, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat.

In some cases, a pharmaceutical composition described herein can be formulated for parenteral (e.g., subcutaneous, intramuscular, intravenous, and intradermal) administration. Compositions suitable for parenteral administration include aqueous and non-aqueous sterile injection solutions that can contain anti-oxidants, buffers, bacteriostats, and solutes which render the formulation isotonic with the blood of the intended recipient; and aqueous and non-aqueous sterile suspensions which may include suspending agents and thickening agents. The formulations can be presented in unit-dose or multi-dose containers, for example, sealed ampules and vials, and may be stored in a freeze dried (lyophilized) condition requiring only the addition of the sterile liquid carrier, for example water for injections, immediately prior to use. Extemporaneous injection solutions and suspensions may be prepared from sterile powders, granules, and tablets.

Any suitable route of administration can be used for a composition containing a nucleic acid encoding a PABPC polypeptide (e.g., a PABPC4 polypeptide or a PABPC1 polypeptide). For example, a pharmaceutical composition containing a nucleic acid construct encoding a PABPC polypeptide can be administered locally (e.g., to the central nervous system or a particular area of the central nervous system, such as the cerebrospinal fluid or the brain), or systemically. In some cases, a nucleic acid encoding a PABPC polypeptide can be administered by direct injection into the brain parenchyma, ventricles, or spinal cord, by intracranial infusion into axonally connected structures of the brain (e.g., the ventral tegmental area or thalamus), or by intranasal administration. In some cases, administration can be parenteral (e.g., by subcutaneous, intrathecal, intramuscular, or intraperitoneal injection, or by intravenous drip). For example, a composition containing a nucleic acid construct encoding a PABPC polypeptide (e.g., a PABPC4 polypeptide or a PABPC1 polypeptide) can be administered systemically by intravenous injection into a mammal (e.g., a human). Administration can be rapid (e.g., by injection) or can occur over a period of time (e.g., by slow infusion or administration of a slow release formulation).

Compositions containing a nucleic acid encoding a PABPC polypeptide (e.g., a PABPC4 polypeptide or a PABPC1 polypeptide) can be administered to a mammal in any amount, at any frequency, and for any duration effective to achieve a desired outcome. For example, a composition containing a nucleic acid encoding a PABPC polypeptide (e.g., a PABPC4 polypeptide or a PABPC1 polypeptide) can be administered in an amount, at a frequency, and for a duration that is sufficient to reduce the level of pathological TDP-43 and/or the level of aggregation of TDP-43 in a mammal (e.g., aggregation of pathological TDP-43 in the brain of the mammal, or in motor neurons of the brain and/or spinal cord of the mammal).

A representative human TDP-43 amino acid sequence is set forth in SEQ ID NO:9: MSEYIRVTEDENDEPIEIPSEDDGTVLLSTVTAQFPGACGLRYRNPVSQCMRGVRLV EGILHAPDAGWGNLVYVVNYPKDNKRKMDETDASSAVKVKRAVQKTSDLIVLGLP WKTTEQDLKEYFSTFGEVLMVQVKKDLKTGHSKGFGFVRFTEYETQVKVMSQRHM IDGRWCDCKLPNSKQSQDEPLRSRKVFVGRCTEDMTEDELREFFSQYGDVMDVFIP KPFRAFAFVTFADDQIAQSLCGEDLIIKGISVHISNAEPKHNSNRQLERSGRFGGNPGG FGNQGGFGNSRGGGAGLGNNQGSNMGGGMNFGAFSINPAMMAAAQAALQSSWG MMGMLASQQNQSGPSGNNQNQGNMQREPNQAFGSGNNSYSGSNSGAAIGWGSAS NAGSGSGFNGGFGSSMDSKSSGWGM (SEQ ID NO:9). A pathological form of TDP-43 can be a C-terminal fragment of TDP-43. In some cases, for example, a pathological TDP-43 polypeptide can consist of amino acids 90-414 of SEQ ID NO:9, amino acids 208-414 of SEQ ID NO:9, about acids 219-414 of SEQ ID NO:9, amino acids 220-414 of SEQ ID NO:9, or amino acids 247-414 of SEQ ID NO:9 (Gendron et al., supra). In some cases, a pathological TDP-43 polypeptide is a full-length or truncated phosphorylated TDP-43 polypeptide. In some cases, for example, pathological TDP-43 can be phosphorylated at one or more of the following amino acids of SEQ ID NO:9: serine 379, serine 403, serine 404, serine 409, and serine 410 (Gendron et al., supra).

In some cases, a composition containing a nucleic acid encoding a PABPC polypeptide (e.g., a PABPC4 polypeptide or a PABPC1 polypeptide) can be administered to a mammal in an amount, at a frequency, and for a duration that is sufficient to reduce one or more symptoms of a TDP-43 proteinopathy in the mammal. In some cases, a composition containing a nucleic acid encoding a PABPC polypeptide (e.g., a PABPC4 polypeptide or a PABPC1 polypeptide) can be administered to a mammal in an amount, at a frequency, and for a duration that is sufficient to promote survival (e.g., to increase the length of overall survival or progression-free survival) of the mammal.

This document also provides methods for using the nucleic acid constructs provided herein to treat a mammal identified as having, being likely to have, or being at increased likelihood of developing, a TDP-43 proteinopathy. As described in the Examples herein, for example, patients with higher levels of PABPC4 exhibited increased survival. In addition, increasing the expression of PABPC4 resulted in reduced levels of TDP-43, including truncated and phosphorylated forms of TDP-43. The methods provided herein can include administering a nucleic acid encoding a PABPC polypeptide (e.g., a PABPC4 polypeptide or a PABPC1 polypeptide) to a mammal having (or suspected to have, or being at increased likelihood to develop) a TDP-43 proteinopathy, such that the level of TDP-43 (e.g., pathological TDP-43) in cells of the mammal is reduced as compared to the level prior to administration of the nucleic acid. In some cases, when a mammal is identified as being at increased risk of developing a TDP-43 proteinopathy, the mammal can be administered a nucleic acid encoding a PABPC polypeptide (e.g., a PABPC4 polypeptide or a PABPC1 polypeptide), such that onset of symptoms is delayed or prevented. Administration of a nucleic acid provided herein to a mammal identified as having (or suspected to have) a TDP-43 proteinopathy can reduce, delay the onset of, or prevent one or more symptoms of the TDP-43 proteinopathy, and/or can extend or increase the likelihood of survival of the mammal.

Any appropriate mammal can be treated as described herein. For example, humans or other primates such as monkeys can be treated to increase the level of a PABPC polypeptide (e.g., a PABPC4 polypeptide or a PABPC1 polypeptide) in cells of the mammal. In some cases, dogs, cats, horses, cows, pigs, sheep, rabbits, mice, and rats can be treated as described herein.

In some cases, a mammal (e.g., a human) identified as having, being likely to have, or being at increased likelihood of developing a TDP-43 proteinopathy (e.g., FTD, ALS, Alzheimer's disease, LBD, or LATE) can be treated by administering a nucleic acid construct encoding a PABPC polypeptide (e.g., a PABPC4 polypeptide or a PABPC1 polypeptide) to the mammal in a manner that reduces the level of TDP-43 in cells of the mammal. A mammal can be identified as having, being likely to have, or being at increased risk of developing a TDP-43 proteinopathy using any appropriate technique. For example, a mammal can be clinically diagnosed as having FTD, ALS, Alzheimer's disease, or LBD. In some cases, while there is currently no diagnostic test specifically to detect TDP-43 proteinopathies in living mammals, a mammal can be identified as being likely to have, or as being at increased risk of developing a TDP-43 proteinopathy based on the presence of clinical signs/symptoms, and/or based on the presence of mutations in genes known to cause TDP-43 pathology. Non-limiting examples of such mutations include repeat expansions within the C9orf72 gene (DeJesus-Hernandez et al., Neuron 2011, 72(2):245-256; and Renton et al., Neuron 2011, 72(2):257-268), as well as mutations in the GRN, VCP, TARDBP, HNRNPA2B1, SETX, DCTN1, ATXN2, UNCI3A, DPP6, TMEM106B, ANG, and/or NIPA1 genes (see, e.g., Baker et al., Nature 2006, 442(7105):916-919; Cruts et al., Nature 2006, 442(7105):920-924; Watts et al., Nat Genet 2004, 36(4):377-381; Sreedharan et al., Science 2008, 319(5870):1668-1672; Kabashi et al., Nat Genet 2008, 40(5):572-574); Kim et al., Nature 2013, 495(7442):467-473; Chen et al., Am J Hum Genet 2004, 74(6):1128-1135; Munch et al., Neurology 2004, 63(4):724-726; Elden et al., Nature 2010, 466(7310):1069-1075; van Es et al., Nat Genet 2009, 41(10):1083-1087; Pottier et al., Acta Neuropathol 2019, 137(6):879-899; and Van Deerlin et al., Nat Genet 2010. 42(3):234-239; Greenway et al., Neurology 2004, 63(10):1936-1938; Blauw et al., Hum Mol Genet 2012, 21(11):2497-2502).

Any appropriate method can be used to deliver a nucleic acid construct encoding a PABPC polypeptide (e.g., a PABPC4 polypeptide or a PABPC1 polypeptide) to a mammal (e.g., to the central nervous system or specifically to motor neurons of the mammal). For example, a nucleic acid construct containing a nucleotide sequence encoding a PABPC polypeptide (e.g., a PABPC4 polypeptide or a PABPC1 polypeptide) can be administered to a mammal in a vector, such as a viral vector. In some cases, a vector for administering a nucleic acid provided herein can be used for transient expression of a PABPC polypeptide (e.g., a PABPC4 polypeptide or a PABPC1 polypeptide). In some cases, a vector for administering a nucleic acid provided herein can be used for stable expression of a PABPC polypeptide (e.g., a PABPC4 polypeptide or a PABPC1 polypeptide). In some cases, where a vector for administering a nucleic acid is to be used for stable expression, the vector can be engineered to integrate the nucleic acid encoding the PABPC polypeptide into the genome. In such cases, any appropriate method can be used to integrate the nucleic acid into the genome of a cell. For example, gene therapy techniques can be used to integrate nucleic acid designed to express a PABPC polypeptide (e.g., a PABPC4 polypeptide or a PABPC1 polypeptide) into the genome of a cell. In some cases, stable expression does not necessarily require integration into the genome. Using adeno-associated virus serotype 9 (AAV9), for example, a nucleic acid can persist on its own in human cells, without integrating into the genome. Non-integrated DNA typically is destroyed as genomic DNA replicates, but in non-dividing cells such as neurons, the DNA can persist indefinitely.

A vector for administering a nucleic acid construct provided herein to cells can be prepared using standard materials (e.g., packaging cell lines, helper viruses, and vector constructs). See, for example, Gene Therapy Protocols (Methods in Molecular Medicine), edited by Jeffrey R. Morgan, Humana Press, Totowa, NJ (2002), and Viral Vectors for Gene Therapy: Methods and Protocols, edited by Curtis A. Machida, Humana Press, Totowa, NJ (2003). Virus-based nucleic acid delivery vectors typically are derived from animal viruses, such as adenoviruses, AAVs, retroviruses, lentiviruses, vaccinia viruses, herpes viruses, and papilloma viruses. In some cases, a nucleic acid construct encoding a PABPC polypeptide (e.g., a PABPC4 polypeptide or a PABPC1 polypeptide) can be delivered to cells using an adeno-associated virus vector, a lentiviral vector, an adenoviral vector, or a herpes simplex virus vector.

In some cases, a virus particle can be used to deliver a nucleic acid construct encoding a PABPC polypeptide (e.g., a PABPC4 polypeptide or a PABPC1 polypeptide) to a mammal. For example, a nucleic acid can be delivered via AAV particles, which are packaged capsid forms of the AAV virus, and can transmit the virus nucleic acid genome to cells. In some cases, a composition containing a virus particle (e.g., an AAV particle) encoded by a viral vector that also encodes a PABPC polypeptide (e.g., a PABPC4 polypeptide or a PABPC1 polypeptide) provided herein can be administered at a concentration from about 10¹⁰ particles/mL to about 105 particles/mL (e.g., from about 10¹⁰ particles/mL to about 10¹¹ particles/mL, from about 10¹⁰ particles/mL to about 10¹² particles/mL, from about 10¹⁰ particles/mL to about 10¹³ particles/mL, from about 10¹¹ particles/mL to about 10¹² particles/mL, from about 10¹¹ particles/mL to about 10¹³ particles/mL, from about 10¹¹ particles/mL to about 10¹⁴ particles/mL, from about 10¹² particles/mL to about 10¹³ particles/mL, from about 10¹² particles/mL to about 10¹⁴ particles/mL, or from about 10¹³ particles/mL to about 10¹⁴ particles/mL). The dose can depend on a number of factors, such as the size (mass) of the mammal, the extent of any side-effects, the particular route of administration, and the like.

In some cases, a nucleic acid construct encoding a PABPC polypeptide (e.g., a PABPC4 polypeptide or a PABPC1 polypeptide) can be administered to a mammal using a non-viral vector. Methods for using non-viral vectors for nucleic acid delivery are described elsewhere. See, for example, Gene Therapy Protocols (Methods in Molecular Medicine), Jeffrey R. Morgan (ed.), Humana Press, Totowa, NJ (2002). For example, a nucleic acid encoding a PABPC polypeptide (e.g., a PABPC4 polypeptide or a PABPC1 polypeptide) can be administered to a mammal by direct injection of nucleic acid molecules (e.g., plasmids), or by administering nucleic acid molecules complexed with lipids, polymers, or nanospheres. In some cases, a nucleic acid designed to express a PABPC polypeptide (e.g., a PABPC4 polypeptide or a PABPC1 polypeptide) can be delivered to cells (e.g., neurons) or tissues or organs via direct injection (e.g., direct injection into the brain parenchyma, ventricles, or spinal cord), intravenous administration, intrathecal administration, intracerebral administration, intraperitoneal administration, intranasal administration, intraparenchymal administration, or oral delivery in nanoparticles and/or drug tablets, capsules, or pills.

Any appropriate amount of a nucleic acid encoding a PABPC polypeptide (e.g., a PABPC4 polypeptide or a PABPC1 polypeptide) can be administered to a mammal (e.g., a human) having a TDP-43 proteinopathy. In some cases, for example, an effective amount of a nucleic acid encoding a PABPC polypeptide (e.g., a PABPC4 polypeptide or a PABPC1 polypeptide) can reduce the level of TDP-43 polypeptides (e.g., full length or truncated forms of TDP-43) in cells (e.g., motor neurons or other neurons in the brain or spinal cord) of a mammal. In some cases, an effective amount of a nucleic acid encoding a PABPC polypeptide (e.g., a PABPC4 polypeptide or a PABPC1 polypeptide) can result in a reduction of one or more symptoms of a TDP-43 proteinopathy in a mammal. In some cases, an effective amount of a nucleic acid encoding a PABPC polypeptide (e.g., a PABPC4 polypeptide or a PABPC1 polypeptide) can delay or prevent the onset of one or more symptoms of a TDP-43 proteinopathy in a mammal. Symptoms of TDP-43 proteinopathies can include, without limitation, dementia, confusion, ataxia, behavioral changes such as poor judgment, apathy, and repetitive compulsive behavior, speech impairment, tremors, rigidity, muscle spasms, poor coordination, swallowing difficulty, muscle weakness, or any combination thereof. In some cases, an effective amount of a nucleic acid encoding a PABPC polypeptide (e.g., a PABPC4 polypeptide or a PABPC1 polypeptide) can extend or increase the likelihood of survival (e.g., overall survival or progression-free survival) of a mammal to which the nucleic acid is administered.

Symptoms can be assessed at any appropriate time after treatment. For example, symptoms can be assessed between 1 day post treatment and 7 days post treatment (e.g., between 1 day and 2 days post treatment, between 1 day and 3 days post treatment, between 1 day and 4 days post treatment, between 2 days and 3 days post treatment, between 2 days and 4 days post treatment, between 2 days and 5 days post treatment, between 3 days and 4 days post treatment, between 3 days and 5 days post treatment, 3 days and 6 days post treatment, between 4 days and 5 days post treatment, between 4 days and 6 days post treatment, between 4 days and 7 days post treatment, between 5 days and 6 days post treatment, between 5 days and 7 days post treatment, or between 6 days and 7 days post treatment). In some cases, symptoms can be assessed between 1 week post treatment and 4 weeks post treatment (e.g., between 1 week and 2 weeks post treatment, between 1 week and 3 weeks post treatment, between 1 week and 4 weeks post treatment, between 2 weeks and 3 weeks post treatment, between 2 weeks and 4 weeks post treatment, or between 3 weeks and 4 weeks post treatment). In some cases, symptoms can be assessed between 1 month post treatment and 12 months post treatment (e.g., between 1 month and 2 months post treatment, between 1 month and 3 months post treatment, between 1 month and 4 months post treatment, between 2 months and 3 months post treatment, between 2 months and 4 months post treatment, between 2 months and 5 months post treatment, between 3 months and 4 months post treatment, between 3 months and 5 months post treatment, between 3 months and 6 months post treatment, between 4 months and 5 months post treatment, between 4 and 6 months post treatment, between 4 months and 7 months post treatment, between 5 months and 6 months post treatment, between 5 months and 7 months post treatment, between 5 months and 8 months post treatment, between 6 months and 7 months post treatment, between 6 months and 8 months post treatment, between 6 months and 9 months post treatment, between 7 months and 8 months post treatment, between 7 months and 9 months post treatment, between 7 months and 10 months post treatment, between 8 months and 9 months post treatment, between 8 months and 10 months post treatment, between 8 months and 11 months post treatment, between 9 months and 10 months post treatment, between 9 months and 11 months post treatment, between 9 months and 12 months post treatment, between 10 months and 11 months post treatment, between 10 months and 12 months post treatment, or between 11 months and 12 months post treatment). In some cases, symptoms can be assessed between 1 year post treatment and about 20 years post treatment (e.g., between 1 year and 5 years post treatment, between 1 year and 10 years post treatment, between 1 year and 15 years post treatment, between 5 years and 10 years post treatment, between 5 years and 15 years post treatment, between 5 years and 20 years post treatment, between 10 years and 15 years post treatment, between 10 years and 20 years post treatment, or between 15 years and 20 years post treatment.

In some cases, a treatment as provided herein can be administered to a mammal (e.g., a human) having a TDP-43 proteinopathy in a single dose, without further administration. In some cases, a treatment as provided herein can be administered to a mammal (e.g., a human) having a TDP-43 proteinopathy at least once daily, or at least once weekly for at least two consecutive days or weeks. In some cases, a treatment as provided herein is administered to a mammal (e.g., a human) having a TDP-43 proteinopathy at least 3, 4, 5, 6, 7, 8, 9, 10, 11, 12, 13, 14, or 15 consecutive days or weeks. In some cases, a treatment as provided herein can be administered to a mammal (e.g., a human) having a TDP-43 proteinopathy at least once daily or at least once weekly for at least 1, 2, 3, 4, 5, 6, 7, 8, 9, 10, 11, or 12 consecutive weeks. In some cases, a treatment as provided herein can be administered to a mammal (e.g., a human) having a TDP-43 proteinopathy at least once daily or at least once weekly for at most 4, 5, 6, 7, 8, 9, 10, 11, 12, 13, 14, 15, 16, 17, 18, 19, or 20 consecutive days or weeks. In some cases, a treatment as provided herein can be administered to a mammal (e.g., a human) having a TDP-43 proteinopathy at least once weekly for at most 1, 2, 3, 4, 5, 6, 7, 8, 9, 10, 11, or 12 consecutive weeks or months. In some cases, a treatment as provided herein can be administered to a mammal (e.g., a human) having a TDP-43 proteinopathy for at least 1, 2, 3, 4, 5, 6, 7, 8, 9, 10, 11, or 12 consecutive months or years, chronically for a subject's entire life span, or an indefinite period of time.

It is to be noted that in some cases, a PABPC polypeptide (e.g., a polypeptide having the amino acid sequence set forth in SEQ ID NO:2, SEQ ID NO:4, SEQ ID NO:6, or SEQ ID NO:8, or an amino acid sequence at least 90% identical to the sequence set forth in SEQ ID NO:2, SEQ ID NO:4, SEQ ID NO:6, or SEQ ID NO:8), can be administered (e.g., intracranially or intrathecally) to a mammal to treat or delay the onset of a TDP-43 proteinopathy.

The invention will be further described in the following examples, which do not limit the scope of the invention described in the claims.

### EXAMPLES

### Example 1—Materials and Methods

Transcriptomics: RNA sequencing (RNAseq) was performed as described elsewhere (Dickson et al., Acta Neuropathol Commun 2019, 7(1):150). In brief, frontal cortex tissue was obtained from patients with a pathological diagnosis of FTLD-TDP with or without ALS who did (N=34) or did not (N=44) carry a C9orf72 repeat expansion, as well as control subjects without neurological diseases (N=24). Total RNA was extracted from frozen brain tissue using the RNEASY® Plus Mini Kit (Qiagen; Hilden, Germany). Libraries were made using the TRUSEQ™ RNA Library Prep Kit v2 (Illumina; San Diego, CA) and sequenced at 10 samples/lane as paired-end 101 base-pair reads on a HiSeq 4000 (Illumina). Raw sequencing reads were aligned to the human reference genome (GRCh38), library quality was assessed, and gene-level expression was quantified. Conditional quantile normalization (CQN) was then used to account for differences in gene counts, gene lengths, and GC content. Genes were retained if their maximum normalized and log 2-transformed reads per kb per million (RPKM) values were above zero. Using linear regression models, source of variation (SOV) analysis was then performed to determine how much variation was explained by potential confounders. The effects of differences in cellular composition between individuals also were assessed using surrogate markers for five major cell types: neurons (ENO2), microglia (CD68), astrocytes (GFAP), oligodendrocytes (OLIG2), and endothelial cells (CD34).

Studies were conducted using samples from all patients (N=78) to determine whether the expression levels of certain genes are associated with clinico-pathological features of diseases characterized by TDP-43 pathology. Residuals were obtained from linear regression models with expression levels as outcome to account for potential confounders (RIN, sex, and plate, either with or without surrogate markers). Cox proportional hazard regression models were run, additionally including disease subgroup (FTLD-TDP and FTLD/ALS) and age at death as potential confounders. Hazard ratios (HRs) and 95% confidence intervals (CIs) were estimated, and deaths of any cause were utilized as survival endpoint. Notably, survival data was only available for a subset of patients (N=71).

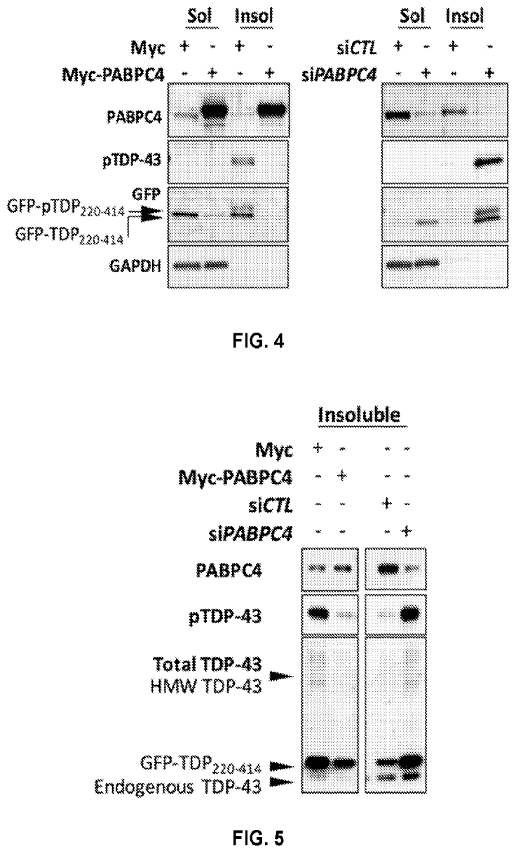

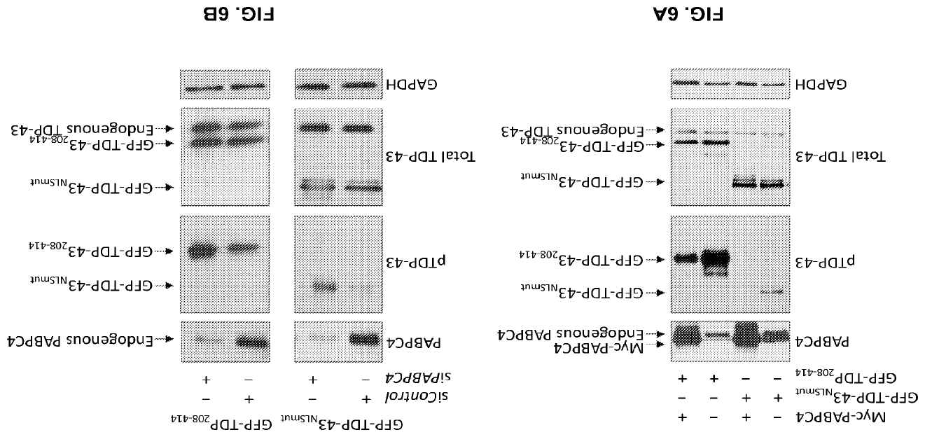

Cell culture studies: To examine the effects of overexpressing PABPC4 on truncated C-terminal TDP-43 fragments (amino acid residues 220-414 of full-length TDP-43; TDP₂₂₀₋₄₁₄), human embryonic kidney 293T (HEK293T) cells (FIG. 4) or human neuroblastoma (M17) cells (FIG. 5) were grown in 12-well plates in Opti-Mem supplemented with 10% fetal bovine serum (FBS) and 1% penicillin/streptomycin. When cells reached 50% confluency, they were co-transfected with either 1 μg/well of plasmid for the expression of myc-tagged PABPC4 (myc-PABPC4) or a control plasmid, and with 1 μg/well of plasmid for the expression of a green fluorescent protein- (GFP-) tagged TDP₂₂₀₋₄₁₄ (GFP-TDP₂₂₀₋₄₁₄) (Zhang et al., Mol Neurodegener 2010, 5:33). Transfections were performed using LIPOFECTAMINE® 2000 (Invitrogen; Carlsbad, CA) according to the manufacturer's instructions. After 48 hours, cells were lysed in buffer (50 mM Tris-HCl, pH 7.4, 1 M NaCl, 1% Triton X-100, 5 mM EDTA) containing PMSF as well as protease and phosphatase inhibitors. After sonication, lysates were centrifuged at 16,000 g at 4° C. for 20 minutes. The supernatant (the Triton X-100 soluble fraction) was saved. The remaining pellets were dissolved in the same buffer as above supplemented with 1% sodium dodecyl sulfate (SDS), sonicated, and centrifuged at 16,000 g at 4° C. for 20 minutes. The resulting supernatant was saved as the Triton X-100 insoluble fraction. The protein concentrations all fractions were determined by BCA assay (Thermo Scientific; Waltham, MA). Samples were heated in Laemmli's buffer and equal amounts of protein were loaded into 10-well 10% Tris-Glycine gels (NOVEX™, Invitrogen). After transfer, blots were blocked with 5% nonfat dry milk in Tris-buffered saline plus 0.1% Triton X-100 (TBST) for 1 hour, and then incubated overnight at 4° C. with antibodies to PABPC4 (A301-466A, Bethyl Laboratories; Montgomery, TX), phosphorylated TDP-43 at serines 409 and 410 (pTDP-43; Rb3655, provided by Leonard Petrucelli, Mayo Clinic group), TDP-43 (Rb5633/5634, provided by Leonard Petrucelli), GFP (Invitrogen, cat. no. A6455), or glyceraldehyde 3-phosphate dehydrogenase (GAPDH) (Meridian, H86504M). Membranes were then washed three times for 10 minutes in TBST and then incubated with donkey anti-rabbit or anti-mouse IgG conjugated to horseradish peroxidase (1:10,000) (Jackson ImmunoResearch; West Grove, PA) for 1 hour. Membranes were washed three times each for 10 minutes, and protein expression was visualized by electrochemiluminescence treatment and exposure to film.

To examine how depleting PABPC4 influences truncated C-terminal TDP-43 fragments, human embryonic kidney 293T (HEK293T) cells or human neuroblastoma (M17) cells were grown in 12-well plates. When cells reached 50% confluency, they were transfected with 20 nM/well small interfering RNA (siRNA) targeting PABPC4 or with a non-targeting control siRNA using LIPOFECT AMINE® RNAiMAX. One day later, cells were transfected with 1 μg/well of plasmid for the expression of GFP-TDP₂₂₀₋₄₁₄ using LIPOFECTAMINE® 2000 (Invitrogen). Cell lysis and Western blotting were performed as described above.

To determine whether PABPC4 or PABPC1 affects the accumulation of other abnormal TDP-43 species in cultured cells, HEK293T cells were co-transfected with plasmids encoding myc-PABPC4 or GFP-PABPC1, or with a control plasmid, and either a plasmid encoding a second C-terminal TDP-43 fragment, GFP-TDP₂₀₈₋₄₁₄ (Zhang et al., supra), or a plasmid encoding full length TDP-43 containing mutations (K82A, R83A, K84A) in the nuclear localization signal (NLS) to cause its cytoplasmic localization (GFP-TDP-43_{NLSmut})(Zhang et al., Proc Natl Acad Sci USA 2009, 106(18):7607-7612). Following transfections, total protein fractions were generated by lysing cells in buffer composed of 50 mM Tris-HCl, pH 7.4, 1 M NaCl, 1% Triton X-100, 5 mM EDTA, 1% SDS, PMSF and protease and phosphatase inhibitors. After sonication, lysates were centrifuged at 16,000 g at 4° C. for 20 minutes and the supernatants were saved. Western blotting was performed as described above with the addition of probing blots with an anti-PABP antibody from Abcam (ab21060).

### Example 2—PABPC4 Expression Levels are Associated with Survival after Onset

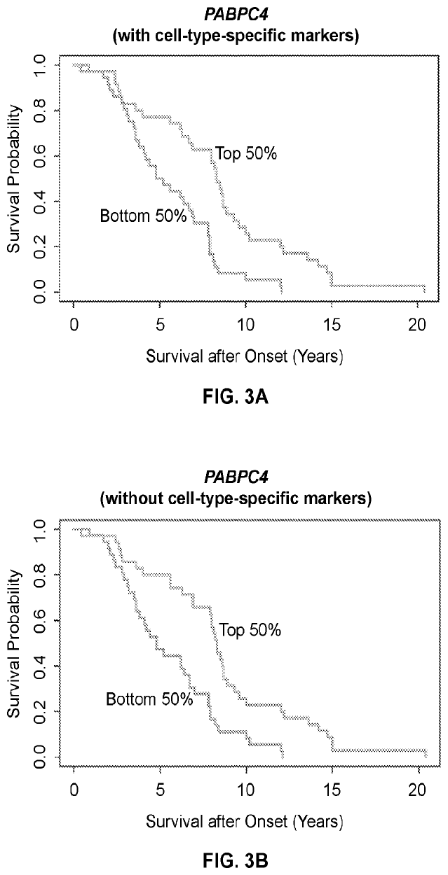

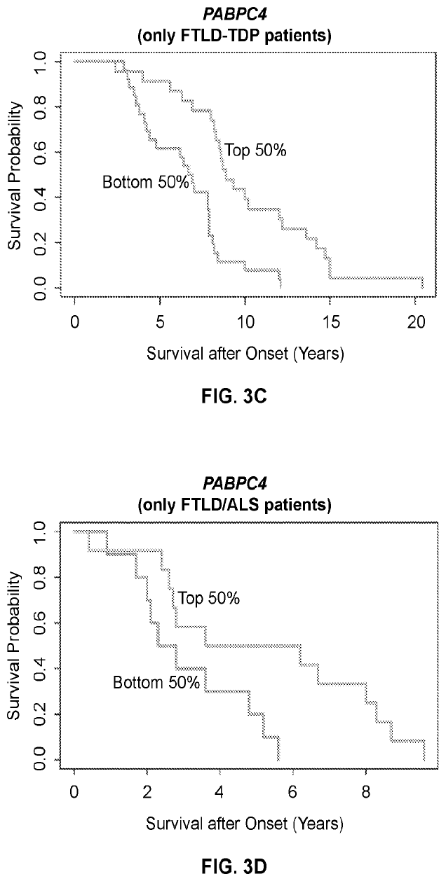

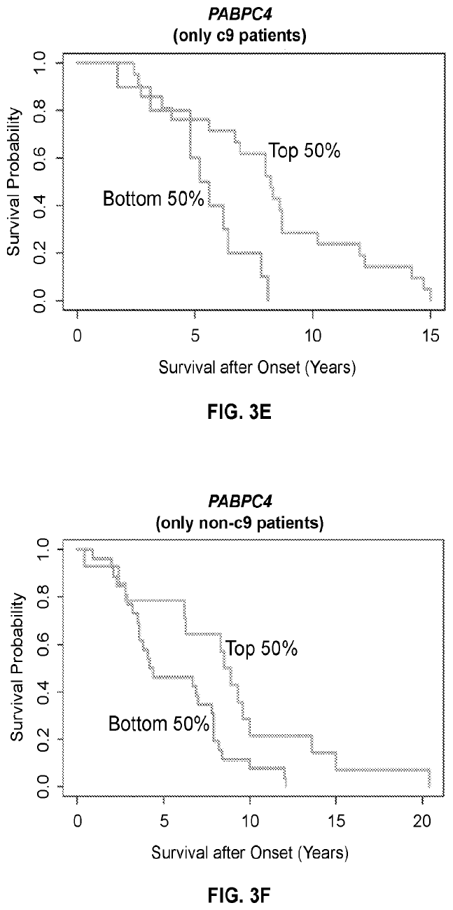

Survival analysis in patients with TDP-43 pathology revealed one gene that remained significant after multiple testing correction: PABPC4 (P=1.2E-07, FDR=0.003; FIGS. 3A-3F). In patients belonging to the bottom 50% of PABPC4 levels, the median survival after onset was 5.0 years (IQR: 3.4-7.8) vs. 8.3 years in the top 50% (IQR: 5.9-10.1), resulting in a hazard ratio (HR) of 0.20 (95% CI: 0.11-0.36). Importantly, a significant correlation between PABPC4 levels measured through RNAseq and quantitative real-time PCR (N=94; P=7.2E-10, r: 0.6) was detected, validating that higher PABPC4 levels were associated with prolonged survival (N=67; P=0.008, HR: 0.49). The RNAseq analysis showed similar findings with and without adjustment for cellular composition (P=3.3E-06, FDR=0.01, HR: 0.26; FIGS. 3A and 3B). Interestingly, the association was more prominent in FTLD-TDP patients (P=5.1E-06; FIG. 3C) than in FTLD/ALS patients (FIG. 3D), and was not driven by the presence of a C9orf72 repeat expansion (P=6.7E-07; FIGS. 3E and 3F). In the cerebellum, no association between PABPC4 and survival was observed (P≥0.33, FDR≥0.80), suggesting that the association might be specific to the severely affected frontal cortex. Of note, although PABPC4 was the top hit of the survival analysis in the frontal cortex, several nominally significant associations were found in this brain region. For instance, an analysis without accounting for cellular composition revealed a similar trend for PABPC1 (P=0.001, FDR=0.11, HR: 0.43), another member of the PABPC family.

### Example 3—PABPC4 and PABPC1 Modulate the Accumulation of Toxic TDP-43 Products

Given that inclusions of TDP-43 are the defining histopathological feature of FTLD-TDP, and given the above-described discovery that PABPC4 was associated with increased survival in patients with FTLD-TDP, further studies were conducted to determine whether PABPC4 influences the accumulation of pathological TDP-43 (C-terminal TDP-43 fragments prone to phosphorylation and aggregation). When myc-PABPC4 was overexpressed in HEK293T cells exogenously expressing GFP-TDP₂₂₀₋₄₁₄, both soluble and insoluble GFP-TDP₂₂₀₋₄₁₄ levels were decreased in comparison to GFP-TDP₂₂₀₋₄₁₄ in cells overexpressing only myc (FIG. 4, left panel). Accumulation of insoluble GFP-TDP₂₂₀₋₄₁₄ phosphorylated at serines 409 and 410 also was markedly decreased in cells expressing myc-PABPC4, compared to cells expressing myc only (FIG. 4, left panel). Conversely, increases in soluble and insoluble GFP-TDP₂₂₀₋₄₁₄, and in insoluble phosphorylated GFP-TDP₂₂₀₋₄₁₄, were observed when PABPC4 was depleted from cells (FIG. 4, right panel). In a similar fashion, overexpressing PABPC4 mitigated GFP-TDP₂₂₀₋₄₁₄ accumulation in M17 cells, while depleting PABPC4 promoted GFP-TDP₂₂₀₋₄₁₄ accumulation (FIG. 5). Further, in addition to influencing levels of monomeric GFP-TDP₂₂₀₋₄₁₄, PABPC4 also modulated levels of insoluble high molecular weight polymeric GFP-TDP₂₂₀₋₄₁₄ (FIG. 5).

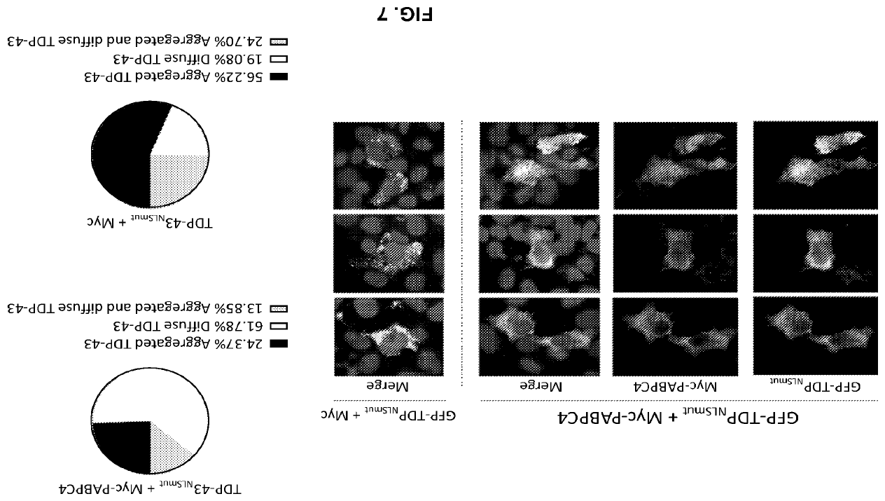

To determine whether PABPC4 reduces the accumulation of other abnormal TDP-43 species in cultured cells, PABPC4 was overexpressed in HEK293T cells along with a second TDP-43 C-terminal fragment (GFP-TDP₂₀₈₋₄₁₄), or a cytoplasmic full-length TDP-43 polypeptide (GFP-TDP-43_{NLSmut}). PABPC4 overexpression was found to attenuate levels of both phosphorylated GFP-TDP₂₀₈₋₄₁₄ and GFP-TDP-43_{NLSmut} (FIG. 6A). Conversely, PABPC4 knockdown augmented levels of phosphorylated GFP-TDP₂₀₈₋₄₁₄ and GFP-TDP-43_{NLSmut} (FIG. 6B), providing further evidence that PABPC4 modulates TDP-43. Additionally, overexpression of Myc-PABPC4 in HEK293T cells co-expressing a GFP tagged TDP-43_{NLSmut} construct significantly reduced the amount of aggregated TDP-43_{NLSmut} in the cytosol when compared with GFP-TDP-43_{NLSmut}-expressing HEK293T cells co-transfected with only myc, supporting the activity of PABPC4 as a regulator of TDP-43 aggregation (FIG. 7). Two individuals independently examined images of GFP-positive cells in a blinded manner, and scored GFP-TDP-43_{NLSmut} as being diffuse, aggregated or both diffuse and aggregated. Between 38-42 GFP-TDP-43_{NLSmut}-positive cells expressing myc-PABPC4 and 38-44 GFP-TDP-43_{NLSmut}-positive cells expressing myc were counted by each blinded scorer.

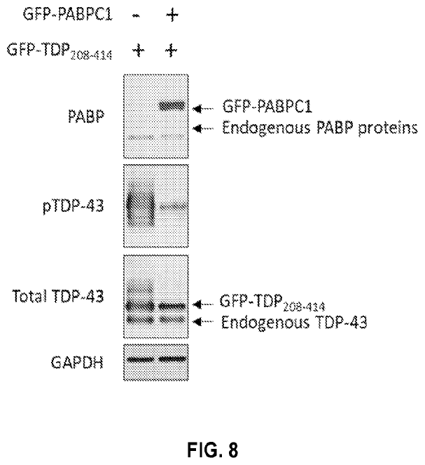

Further, since a nominally significant association was observed between PABPC1 and longer survival in patients with TDP-43 pathology, studies were conducted to determine whether PABPC1, like PABPC4, could modify accumulation of aberrant TDP-43. These studies revealed that PABPC1 overexpression attenuated levels of phosphorylated GFP-TDP₂₀₈₋₄₁₄ (FIG. 8).

Example 4—Generation of Adeno-associated viral (AAV) vectors A PABPC4 (NM_001135653; SEQ ID NO:10) Human Tagged ORF Clone (cat #RC226744, Origene) was designed in a pCMV6-Entry vector. To remove the 3′ myc-DDK tag, PABPC4 was excised out of the pCMV vector and subcloned into an AAV expression vector (pAM/CBA-pl-WPRE-BGH; Fitzsimons et al., Methods. 2002, 28(2):227-236; SEQ ID NO:11) containing inverted repeats of serotype 2. To generate AAV for injecting AAV-PABPC4 or AAV-empty vector, particles were packaged into AAV serotype 9 capsids and purified as described elsewhere (Zolotukhin et al., Gene Ther. 1999, 6:973-985). Briefly, the AAV expression vectors were co-transfected with helper plasmids into HEK293T cells. Cells were harvested 72 hours later, treated with 50 units/ml Benzonase (Sigma Aldrich), and lysed by freeze thaw with 0.5% sodium deoxycholate. The virus was purified from these lysates using a discontinuous iodixanol gradient, and the genomic titer of each virus was determined by qPCR. Viruses were diluted to a standard titer of 1E13 using phosphate-buffered saline (PBS), aliquoted, and frozen prior to injection.

### Example 5—PABPC4 Expression Modulates TDP-43 Aggregation in a Mouse Model of TDP-43 Pathology

Study overview: To test PABPC4 gene therapy in vivo, studies are conducted to evaluate whether overexpressing PABPC4 in the central nervous system of mice that develop cytoplasmic TDP-43 pathology, motor deficits, and early death can abrogate these aberrant features. These studies utilize rNLS8 mice, which express human TDP-43 with a mutated nuclear localization signal (hTDP-43-ΔNLS) under the control of the neurofilament heavy chain (NEFH) promoter in the absence of doxycycline (Dox). These mice recapitulate salient features of human TDP-43 proteinopathies, such as TDP-43 pathology, neuron loss, brain atrophy, motor impairments, and early death (Walker et al., Acta Neuropathol. 2015, 130:643-660). To generate rNLS8 mice, monogenic NEFH-tTA line 8 mice (The Jackson Laboratory, strain #025397) are crossed with tetO-hTDP-43-ΔNLS line 4 mice (The Jackson Laboratory, strain #014560).

Two studies are conducted, each using separate cohorts of mice, with the purpose of examining whether PABPC4 overexpression in the central nervous system of rNLS8 mice attenuates the early accumulation, phosphorylation, and aggregation of hTDP-43-ΔNLS, neuron loss and motor phenotypes (study 1), and whether PABPC4 overexpression in the central nervous system slows neurodegeneration and disease progression (study 2).

For each study, postnatal day 0 pups from 24 litters from monogenic NEFH-tTA and tetO-hTDP-43-ΔNLS mouse crosses are transduced; specifically, 12 litters are transduced to express PABPC4, and 12 litters are transduced with empty vector. Since each litter is comprised of non-transgenic, NEFH-tTA monogenic, hTDP-43ΔNLS monogenic, and biogenic rNLS8 pups, about 15 rNLS8 pups are administered intracerebroventricular (ICV) injections of AAV vectors for the expression of PABPC4, and about 15 rNLS8 pups are transduced with empty vector (based on a yield of five mice per litter).

All procedures involving mice are performed in accordance with the National Institutes of Health Guide for Care and Use of Experimental Animals and approved by the Mayo Clinic Institutional Animal Care and Use Committee (IACUC). Mice are maintained on a 12-hour light/dark cycle in standard housing. Both male and female mice are included in each experimental cohort.

When in their home cage, mice have access to chow (standard chow or Dox-containing chow to induce or repress transgene expression, respectively) and water ad libitum. Breeding mice and the resulting pups are fed Dox chow, with the pups being maintained on Dox chow until they are about five weeks of age. At that time, Dox is removed to allow hTDP-43-ΔNLS expression in rNLS8 mice. Thereafter, the weight of mice, and whether they develop clasping and tremor abnormalities, is logged weekly or more frequently until mice are sacrificed.

In study 1, mice undergo behavioral assessments two weeks after hTDP-43-ΔNLS expression is induced. About one week later, mice are euthanized, and blood, brain, and spinal cord are collected for biochemical and histochemical evaluations as described below. In study 2, mice also undergo behavioral assessments two weeks after hTDP-43-ΔNLS expression is induced. Thereafter, mice are monitored closely and, when they meet humane endpoints (a proxy for survival), they are euthanized. Blood, brain and spinal cord are collected for biochemical and histochemical evaluations as described below.

Intracerebroventricular (ICV) delivery of adeno-associated viral (AAV) vector: ICV injections of AAV are carried-out as described elsewhere (Chew et al., Science 2015, 348(6239):1151-1154; and Chew et al., Mol Neurodegener. 2019, 14(1):9). Briefly, post-natal day zero pups are cryoanesthetized on ice. Two microliters (1E13 viral genomes/μl) of the desired AAV solution are manually injected into each lateral ventricle (just posterior to bregma and 2 mm lateral to the midline) using a 32-gauge needle (product #7803-04, 0.5 in. custom length, point style 4, 12 degrees, Hamilton Company) fitted to a 10 μl syringe (Hamilton Company). Following injection, pups are allowed to recover on a heated pad before being returned to their home cage.

Behavioral Tests and Observations

Open Field Test: This protocol begins with a one-hour acclimation of the mice in the room in which they are tested. The mice are placed in the activity chamber for a specified time period (e.g., 10-minute intervals). Activity levels and movement in three dimensions are recorded by the activity system, and are analyzed for evidence of hyperactivity, hypoactivity, anxiety, explorative behaviors, and stereotyped rotation. The dimensions of the Open Field Test box are 40 cm×40 cm×30 cm (W×L×H).

Hanging wire test: A 55 cm wide, 2 mm thick wire is secured tightly to two vertical stands. The wire is maintained 35 cm above a layer of bedding material to prevent injury to the animal when it falls. The mice are picked up by the tail and brought close to the wire so that their forelimbs can grip the wire. The ability of each mouse to suspend itself on the rod, and the number of falls from the wire within 2 minutes are assessed.

Rotarod test: To test motor learning and coordination, mice are placed on an accelerating rotarod apparatus (Ugo Basile) for 16 trials (4 trials on four consecutive days) with a 30- to 60-minute rest interval between trials. Each trial is conducted for a maximum of 15 minutes, during which the rod (which is about 6 inches off the ground) accelerates linearly from 4 to 40 rpm. The amount of time for each mouse to fall from the rod is recorded for each trial. Soft padding material is placed under the rod to cushion the falls.

Hindlimb clasping observations: To observe hindlimb clasping, mice are suspended by the tail about 30 cm above the cage and slowly lowered. The presence of both hindlimbs held together within 5 seconds of being raised and maintained for −30 seconds is recorded as a positive response.

Tremor observations: To observe tremor, mice are held on their backs in the palm of the observer's hand, gripped gently between thumb and index finger, and forelimb and hindlimb movements are observed for 30 seconds. The presence of fast fine tremor at any point in this observation period is recorded as a positive response.

Blood collection and tissue processing: Mice are anesthetized with ketamine/xylazine, and a cardiac puncture will be performed to collect blood (in EDTA tubes) followed by transcardial perfusion with saline. Blood in EDTA tubes is centrifuged to obtain plasma. Brains and spinal cord are harvested, and brains are cut sagittally across the midline. Sagittal half brains and spinal cords are immersion fixed in in 4% paraformaldehyde, embedded in paraffin, sectioned (5 μm thick), and mounted on glass slides for immunohistochemical or immunofluorescence staining. The other half brains are dissected (cortex, hippocampus, subcortex, midbrain, brainstem, and cerebellum) and frozen separately.

Immunohistochemical and immunofluorescence staining: Fixed sagittal half brain and spinal cord sections are deparaffinized in xylene and rehydrated through a series of ethanol solutions, followed by washing in dH₂O. For immunohistochemistry, antigen retrieval is performed by steaming slides in dH₂O for 30 minutes (or, when appropriate, in Tris-EDTA, pH 9.0 or in 10 mM sodium citrate, 0.05% Tween-20, pH 6.0), followed by a 5-minute incubation in Dako Peroxidase Block (52001, Dako) to block endogenous peroxidase activity. Slides are blocked with Dako Protein Block Serum-Free (X0909, Dako) for 1 hour, and incubated for 45-60 minutes with antibodies for the detection of proteins of interest, such as TDP-43 (12892-1-AP, Proteintech; 2E2-D3, Novus Biologicals), phosphorylated TDP-43 (CAC-TIP-PTD-M01, Cosmo Bio), PABPC4 (HPA027301 and HPA056496, Atlas Antibodies; PA5-66018, Invitrogen), GFAP (z0334, Dako), and the neuron marker, NeuN (MAB377, Chemicon International). Subsequently, tissue sections are washed and incubated for 30 minutes in Dako Envision-Plus anti-rabbit (K4003, Dako) or anti-mouse (K4001, Dako) labeled HRP polymer. Peroxidase labeling is visualized with the Liquid DAB+Substrate Chromogen System (K3468, Dako). Slides are scanned with a ScanScope AT2 (Leica Biosystems), and representative images are taken with ImageScope software (v12.1; Leica Biosystems) for analysis of TDP-43, phosphorylated TDP-43, PABCP4, GFAP, or NeuN-immunopositive neurons. For immunofluorescence staining, deparaffinized and rehydrated sections are steamed for 30 minutes in Dako antigen retrieval solution, blocked with Dako All Purpose Blocker for 1 hour, and incubated with primary antibodies for the detection of proteins of interest. After washing, sections are incubated with species-appropriate Alexa Fluor secondary antibodies (Molecular Probes) for 2 hours. Hoechst 33258 (Thermo Fisher Scientific) is used to stain cellular nuclei. Images are obtained on a Zeiss LSM 880 laser scanning confocal microscope.

Biochemical analyses and immunoblotting: Frozen brain tissues are thawed on ice and subjected to RIPA-soluble and urea-soluble fractionation as described elsewhere (Walker et al., supra). These protein fractions are analyzed by Western blotting to measure soluble and insoluble proteins of interest, including TDP-43 (12892-1-AP, Proteintech; 2E2-D3, Novus Biologicals), phosphorylated TDP-43 (CAC-TIP-PTD-M01, Cosmo Bio), PABPC4 (A301-466A, Bethyl Lab), and GAPDH (H86504M, Meridian). In brief, lysates are diluted with 2×SDS-loading buffer at a 1:1 ratio (v/v). RIPA-soluble fractions, but not urea-soluble fractions, are heated at 95° C. for 5 minutes. Equal amounts of RIPA-soluble protein or urea-soluble protein are loaded into Novex WedgeWell 10% Tris-Glycine 10- or 15-well gels (XP00100BOX, XP00105BOX, Invitrogen). After transferring proteins to nitrocellulose membranes (45-004-012, GE), membranes are blocked with 5% nonfat dry milk in TRIS-buffer saline (TBS) plus 0.1% Triton X-100 (TBST) for 1 hour, and then incubated with primary antibodies overnight at 4° C. Membranes are washed in TBST and incubated with donkey anti-rabbit or anti-mouse IgG antibodies conjugated to horseradish peroxidase (Jackson ImmunoResearch) for 1 hour. Protein expression is visualized by enhanced chemiluminescence treatment using the Amersham ImageQuant 800. The intensity of bands is quantified by FUJI FILM MultiGauge Software.

Detection of neurofilament light in plasma: Concentrations of neurofilament light (NfL), a marker of neuronal injury, are determined using the NF-Light digital immunoassay (103186, Quanterix) run on the automated HD-1 Analyzer (Quanterix) per the manufacturer's protocol and as described elsewhere (Cook et al., Sci Translat Med. 2020, 12(559):eabb3774). In brief, plasma samples are diluted 1:4 at the bench, and subsequently transferred to 96-well plates along with calibrators, two quality control samples, and five inter-assay controls with a range of known NfL concentrations. Concentrations in pg/ml are interpolated from the standard curve using a 4-parameter logistic curve fit (1/y2 weighted).

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. A method for treating a mammal identified as having or being likely to have a TDP-43 proteinopathy, said method comprising administering to said mammal a nucleic acid construct comprising a nucleotide sequence encoding a polyadenylate-binding protein 4 (PABPC4) polypeptide or a polyadenylate-binding protein 1 (PABPC1) polypeptide, wherein said administering is effective to reduce one or more symptoms of said TDP-43 proteinopathy.

2. The method of claim 1, wherein said nucleotide sequence encodes a PABPC4 polypeptide comprising an amino acid sequence having at least 90% sequence identity to SEQ ID NO:2, SEQ ID NO:4, SEQ ID NO:6, or SEQ ID NO:8.

3-5. (canceled)

6. The method of claim 2, wherein said nucleotide sequence has at least 90% sequence identity to SEQ ID NO:1, SEQ ID NO:3, SEQ ID NO:5, or SEQ ID NO:7.

7-15. (canceled)

16. The method of claim 1, wherein said TDP-43 proteinopathy comprises frontotemporal dementia (FTD), amyotrophic lateral sclerosis (ALS), Alzheimer's disease, Lewy body dementia (LBD), or limbic-predominant age-related TDP-43 encephalopathy (LATE).

17. The method of claim 1, wherein said nucleotide sequence encoding said PABPC4 polypeptide is operably linked to a promoter.

18-21. (canceled)

22. The method of claim 1, wherein said nucleic acid construct is within a viral vector.

23. (canceled)

24. The method of claim 1, wherein said administering comprises delivering said nucleic acid construct to cells in the brain of said mammal or to cells in the spinal cord of said mammal.

25-26. (canceled)

27. A method for reducing accumulation of a pathologic TDP-43 polypeptide within neuronal cells of a mammal identified as having, being likely to have, or being at increased risk of developing a TDP-43 proteinopathy, wherein said method comprises administering to said mammal a nucleic acid construct comprising a nucleotide sequence encoding a PABPC4 polypeptide or a PABPC1 polypeptide.

28. The method of claim 27, wherein said pathologic TDP-43 polypeptide is a TDP-43₂₀₈₋₄₁₄ fragment, a TDP-43₂₂₀₋₄₁₄ fragment, or a phosphorylated TDP-43 polypeptide.

29. The method of claim 27, wherein said nucleotide sequence encodes a PABPC4 polypeptide comprising an amino acid sequence having at least 90% sequence identity to SEQ ID NO:2, SEQ ID NO:4, SEQ ID NO:6, or SEQ ID NO:8.

30-32. (canceled)

33. The method of claim 29, wherein said nucleotide sequence has at least 90% sequence identity to SEQ ID NO:1, SEQ ID NO:3, SEQ ID NO:5, or SEQ ID NO:7.

34-42. (canceled)

43. The method of claim 27, wherein said TDP-43 proteinopathy comprises FTD, ALS, Alzheimer's disease, LBD, or LATE.

44. The method of claim 27, wherein said nucleotide sequence encoding said PABPC4 polypeptide is operably linked to a promoter.

45-48. (canceled)

49. The method of claim 27, wherein said nucleic acid construct is within a viral vector.

50. (canceled)

51. The method of claim 27, wherein said administering comprises delivering said nucleic acid construct to cells in the brain of said mammal or to cells in the spinal cord of said mammal.

52-53. (canceled)

54. A method for reducing one or more symptoms of a TDP-43 proteinopathy in a mammal, said method comprising administering to said mammal a nucleic acid construct comprising a nucleotide sequence encoding a PABPC4 polypeptide or PABPC1 polypeptide, wherein said nucleic acid construct is administered in an amount effective to reduce one or more symptoms of said TDP-43 proteinopathy in said mammal.

55. The method of claim 54, wherein said nucleotide sequence encodes a PABPC4 polypeptide comprising an amino acid sequence having at least 90% sequence identity to SEQ ID NO:2, SEQ ID NO:4, SEQ ID NO:6, or SEQ ID NO:8.

56-58. (canceled)

59. The method of claim 55, wherein said nucleotide sequence has at least 90% sequence identity to SEQ ID NO:1, SEQ ID NO:3, SEQ ID NO:5, or SEQ ID NO:7.

60-68. (canceled)

69. The method of claim 54, wherein said TDP-43 proteinopathy comprises FTD, ALS, Alzheimer's disease, LBD, or LATE.

70. The method of claim 54, wherein said nucleotide sequence encoding said PABPC4 polypeptide is operably linked to a promoter.

71-74. (canceled)

75. The method of claim 54, wherein said nucleic acid construct is within a viral vector.

76. (canceled)

77. The method of claim 54, wherein said administering comprises delivering said nucleic acid construct to cells in the brain of said mammal or to cells in the spinal cord of said mammal.

78-106. (canceled)


SEQUENCE LISTING (11 sequences)

SEQ ID NO: 1 - DNA, 1983 bp, Homo sapiens
atgaacgctgcggccagcagctaccccatggcctccctgtacgtgggcgacctgcattcggacgtcaccgaggccatgctgtacgaaaagttcagccccgcggggcctgtgctgtccatccgggtctgccgcgatatgatcacccgccgctccctgggctatgcctacgtcaacttccagcagccggccgacgctgagcgggctttggacaccatgaactttgatgtgattaagggaaagccaatccgcatcatgtggtctcagagggatccctctttgagaaaatctggtgtgggaaacgtcttcatcaagaacctggacaaatctatagataacaaggcactttatgatactttttctgcttttggaaacatactgtcctgcaaggtggtgtgtgatgagaacggctctaagggttatgcctttgtccacttcgagacccaagaggctgccgacaaggccatcgagaagatgaatggcatgctcctcaatgaccgcaaagtatttgtgggcagattcaagtctcgcaaagagcgggaagctgagcttggagccaaagccaaggaattcaccaatgtttatatcaaaaactttggggaagaggtggatgatgagagtctgaaagagctattcagtcagtttggtaagaccctaagtgtcaaggtgatgagagatcccaatgggaaatccaaaggctttggctttgtgagttacgaaaaacacgaggatgccaataaggctgtggaagagatgaatggaaaagaaataagtggtaaaatcatatttgtaggccgtgcacaaaagaaagtagaacggcaggcagagttaaaacggaaatttgaacagttgaaacaggagagaattagtcgatatcagggggtgaatctctacattaagaacttggatgacactattgatgatgagaaattaaggaaagaattttctccttttggatcaattaccagtgctaaggtaatgctggaggatggaagaagcaaagggtttggcttcgtctgcttctcatctcctgaagaagcaaccaaagcagtcactgagatgaatggacgcattgtgggctccaagccactatatgttgccctggcccagaggaaggaagagagaaaggctcacctgaccaaccagtatatgcaacgagtggctggaatgagagcacttcctgccaatgccatcttaaatcagttccagcctgcagcgggtggctactttgtgccagcagtcccacaggctcagggaaggcctccatattatacacctaaccagttagcacagatgaggcctaatccacgctggcagcaaggtgggagacctcaaggcttccaaggaatgccaagtgctatacgccagtctgggcctcgtccaactcttcgccatctggctccaactggtaatgctccggcctctcgtggcctccctactaccactcagagagtcgggtctgagtgcccggaccgcttggctatggactttggtggggctggtgccgcccagcaagggctgactgacagctgccagtctggaggcgttcccacagctgtgcagaacttagcgccacgcgctgctgttgctgctgctgctccccgggctgttgccccctacaaatacgcctccagtgtccgcagccctcatcctgccatacagcctctgcaggcaccccagcctgcggtccatgtgcaggggcaggagccactgactgcctccatgctggctgcagcacccccccaggaacagaagcagatgctgggagaacgcttgttcccactcatccaaacaatgcattcaaatctggctgggaagatcacgggaatgctgctggagatagacaactctgagctgctgcacatgttagagtcccccgagtctctccgctccaaggtggatgaagctgtagcagttctacaggctcatcatgccaagaaagaagctgcccagaaggtgggcgctgttgctgctgctacctcttag

SEQ ID NO: 2 - protein, 660 aa, Homo sapiens
MNAAASSYPMASLYVGDLHSDVTEAMLYEKFSPAGPVLSIRVCRDMITRRSLGYAYVNFQQPADAERALDTMNFDVIKGKPIRIMWSQRDPSLRKSGVGNVFIKNLDKSIDNKALYDTFSAFGNILSCKVVCDENGSKGYAFVHFETQEAADKAIEKMNGMLLNDRKVFVGRFKSRKEREAELGAKAKEFTNVYIKNFGEEVDDESLKELFSQFGKTLSVKVMRDPNGKSKGFGFVSYEKHEDANKAVEEMNGKEISGKIIFVGRAQKKVERQAELKRKFEQLKQERISRYQGVNLYIKNLDDTIDDEKLRKEFSPFGSITSAKVMLEDGRSKGFGFVCFSSPEEATKAVTEMNGRIVGSKPLYVALAQRKEERKAHLTNQYMQRVAGMRALPANAILNQFQPAAGGYFVPAVPQAQGRPPYYTPNQLAQMRPNPRWQQGGRPQGFQGMPSAIRQSGPRPTLRHLAPTGNAPASRGLPTTTQRVGSECPDRLAMDFGGAGAAQQGLTDSCQSGGVPTAVQNLAPRAAVAAAAPRAVAPYKYASSVRSPHPAIQPLQAPQPAVHVQGQEPLTASMLAAAPPQEQKQMLGERLFPLIQTMHSNLAGKITGMLLEIDNSELLHMLESPESLRSKVDEAVAVLQAHHAKKEAAQKVGAVAAATS

SEQ ID NO: 3 - DNA, 1935 bp, Homo sapiens
atgaacgctgcggccagcagctaccccatggcctccctgtacgtgggcgacctgcattcggacgtcaccgaggccatgctgtacgaaaagttcagccccgcggggcctgtgctgtccatccgggtctgccgcgatatgatcacccgccgctccctgggctatgcctacgtcaacttccagcagccggccgacgctgagcgggctttggacaccatgaactttgatgtgattaagggaaagccaatccgcatcatgtggtctcagagggatccctctttgagaaaatctggtgtgggaaacgtcttcatcaagaacctggacaaatctatagataacaaggcactttatgatactttttctgcttttggaaacatactgtcctgcaaggtggtgtgtgatgagaacggctctaagggttatgcctttgtccacttcgagacccaagaggctgccgacaaggccatcgagaagatgaatggcatgctcctcaatgaccgcaaagtatttgtgggcagattcaagtctcgcaaagagcgggaagctgagcttggagccaaagccaaggaattcaccaatgtttatatcaaaaactttggggaagaggtggatgatgagagtctgaaagagctattcagtcagtttggtaagaccctaagtgtcaaggtgatgagagatcccaatgggaaatccaaaggctttggctttgtgagttacgaaaaacacgaggatgccaataaggctgtggaagagatgaatggaaaagaaataagtggtaaaatcatatttgtaggccgtgcacaaaagaaagtagaacggcaggcagagttaaaacggaaatttgaacagttgaaacaggagagaattagtcgatatcagggggtgaatctctacattaagaacttggatgacactattgatgatgagaaattaaggaaagaattttctccttttggatcaattaccagtgctaaggtaatgctggaggatggaagaagcaaagggtttggcttcgtctgcttctcatctcctgaagaagcaaccaaagcagtcactgagatgaatggacgcattgtgggctccaagccactatatgttgccctggcccagaggaaggaagagagaaaggctcacctgaccaaccagtatatgcaacgagtggctggaatgagagcacttcctgccaatgccatcttaaatcagttccagcctgcagcgggtggctactttgtgccagcagtcccacaggctcagggaaggcctccatattatacacctaaccagttagcacagatgaggcctaatccacgctggcagcaaggtgggagacctcaaggcttccaaggaatgccaagtgctatacgccagtctgggcctcgtccaactcttcgccatctggctccaactgggtctgagtgcccggaccgcttggctatggactttggtggggctggtgccgcccagcaagggctgactgacagctgccagtctggaggcgttcccacagctgtgcagaacttagcgccacgcgctgctgttgctgctgctgctccccgggctgttgccccctacaaatacgcctccagtgtccgcagccctcatcctgccatacagcctctgcaggcaccccagcctgcggtccatgtgcaggggcaggagccactgactgcctccatgctggctgcagcacccccccaggaacagaagcagatgctgggagaacgcttgttcccactcatccaaacaatgcattcaaatctggctgggaagatcacgggaatgctgctggagatagacaactctgagctgctgcacatgttagagtcccccgagtctctccgctccaaggtggatgaagctgtagcagttctacaggctcatcatgccaagaaagaagctgcccagaaggtgggcgctgttgctgctgctacctcttag

SEQ ID NO: 4 - protein, 644 aa, Homo sapiens
MNAAASSYPMASLYVGDLHSDVTEAMLYEKFSPAGPVLSIRVCRDMITRRSLGYAYVNFQQPADAERALDTMNFDVIKGKPIRIMWSQRDPSLRKSGVGNVFIKNLDKSIDNKALYDTFSAFGNILSCKVVCDENGSKGYAFVHFETQEAADKAIEKMNGMLLNDRKVFVGRFKSRKEREAELGAKAKEFTNVYIKNFGEEVDDESLKELFSQFGKTLSVKVMRDPNGKSKGFGFVSYEKHEDANKAVEEMNGKEISGKIIFVGRAQKKVERQAELKRKFEQLKQERISRYQGVNLYIKNLDDTIDDEKLRKEFSPFGSITSAKVMLEDGRSKGFGFVCFSSPEEATKAVTEMNGRIVGSKPLYVALAQRKEERKAHLTNQYMQRVAGMRALPANAILNQFQPAAGGYFVPAVPQAQGRPPYYTPNQLAQMRPNPRWQQGGRPQGFQGMPSAIRQSGPRPTLRHLAPTGSECPDRLAMDFGGAGAAQQGLTDSCQSGGVPTAVQNLAPRAAVAAAAPRAVAPYKYASSVRSPHPAIQPLQAPQPAVHVQGQEPLTASMLAAAPPQEQKQMLGERLFPLIQTMHSNLAGKITGMLLEIDNSELLHMLESPESLRSKVDEAVAVLQAHHAKKEAAQKVGAVAAATS

SEQ ID NO: 5 - DNA, 1896 bp, Homo sapiens
atgaacgctgcggccagcagctaccccatggcctccctgtacgtgggcgacctgcattcggacgtcaccgaggccatgctgtacgaaaagttcagccccgcggggcctgtgctgtccatccgggtctgccgcgatatgatcacccgccgctccctgggctatgcctacgtcaacttccagcagccggccgacgctgagcgggctttggacaccatgaactttgatgtgattaagggaaagccaatccgcatcatgtggtctcagagggatccctctttgagaaaatctggtgtgggaaacgtcttcatcaagaacctggacaaatctatagataacaaggcactttatgatactttttctgcttttggaaacatactgtcctgcaaggtggtgtgtgatgagaacggctctaagggttatgcctttgtccacttcgagacccaagaggctgccgacaaggccatcgagaagatgaatggcatgctcctcaatgaccgcaaagtatttgtgggcagattcaagtctcgcaaagagcgggaagctgagcttggagccaaagccaaggaattcaccaatgtttatatcaaaaactttggggaagaggtggatgatgagagtctgaaagagctattcagtcagtttggtaagaccctaagtgtcaaggtgatgagagatcccaatgggaaatccaaaggctttggctttgtgagttacgaaaaacacgaggatgccaataaggctgtggaagagatgaatggaaaagaaataagtggtaaaatcatatttgtaggccgtgcacaaaagaaagtagaacggcaggcagagttaaaacggaaatttgaacagttgaaacaggagagaattagtcgatatcagggggtgaatctctacattaagaacttggatgacactattgatgatgagaaattaaggaaagaattttctccttttggatcaattaccagtgctaaggtaatgctggaggatggaagaagcaaagggtttggcttcgtctgcttctcatctcctgaagaagcaaccaaagcagtcactgagatgaatggacgcattgtgggctccaagccactatatgttgccctggcccagaggaaggaagagagaaaggctcacctgaccaaccagtatatgcaacgagtggctggaatgagagcacttcctgccaatgccatcttaaatcagttccagcctgcagcgggtggctactttgtgccagcagtcccacaggctcagggaaggcctccatattatacacctaaccagttagcacagatgaggcctaatccacgctggcagcaaggtgggagacctcaaggcttccaaggaatgccaagtgctatacgccagtctgggcctcgtccaactcttcgccatctggctccaactggtaatgctccggcctctcgtggcctccctactaccactcagagagtcggcgttcccacagctgtgcagaacttagcgccacgcgctgctgttgctgctgctgctccccgggctgttgccccctacaaatacgcctccagtgtccgcagccctcatcctgccatacagcctctgcaggcaccccagcctgcggtccatgtgcaggggcaggagccactgactgcctccatgctggctgcagcacccccccaggaacagaagcagatgctgggagaacgcttgttcccactcatccaaacaatgcattcaaatctggctgggaagatcacgggaatgctgctggagatagacaactctgagctgctgcacatgttagagtcccccgagtctctccgctccaaggtggatgaagctgtagcagttctacaggctcatcatgccaagaaagaagctgcccagaaggtgggcgctgttgctgctgctacctcttag

SEQ ID NO: 6 - protein, 631 aa, Homo sapiens
MNAAASSYPMASLYVGDLHSDVTEAMLYEKFSPAGPVLSIRVCRDMITRRSLGYAYVNFQQPADAERALDTMNFDVIKGKPIRIMWSQRDPSLRKSGVGNVFIKNLDKSIDNKALYDTFSAFGNILSCKVVCDENGSKGYAFVHFETQEAADKAIEKMNGMLLNDRKVFVGRFKSRKEREAELGAKAKEFTNVYIKNFGEEVDDESLKELFSQFGKTLSVKVMRDPNGKSKGFGFVSYEKHEDANKAVEEMNGKEISGKIIFVGRAQKKVERQAELKRKFEQLKQERISRYQGVNLYIKNLDDTIDDEKLRKEFSPFGSITSAKVMLEDGRSKGFGFVCFSSPEEATKAVTEMNGRIVGSKPLYVALAQRKEERKAHLTNQYMQRVAGMRALPANAILNQFQPAAGGYFVPAVPQAQGRPPYYTPNQLAQMRPNPRWQQGGRPQGFQGMPSAIRQSGPRPTLRHLAPTGNAPASRGLPTTTQRVGVPTAVQNLAPRAAVAAAAPRAVAPYKYASSVRSPHPAIQPLQAPQPAVHVQGQEPLTASMLAAAPPQEQKQMLGERLFPLIQTMHSNLAGKITGMLLEIDNSELLHMLESPESLRSKVDEAVAVLQAHHAKKEAAQKVGAVAAATS

SEQ ID NO: 7 - DNA, 1911 bp, Homo sapiens
atgaaccccagtgcccccagctaccccatggcctcgctctacgtgggggacctccaccccgacgtgaccgaggcgatgctctacgagaagttcagcccggccgggcccatcctctccatccgggtctgcagggacatgatcacccgccgctccttgggctacgcgtatgtgaacttccagcagccggcggacgcggagcgtgctttggacaccatgaattttgatgttataaagggcaagccagtacgcatcatgtggtctcagcgtgatccatcacttcgcaaaagtggagtaggcaacatattcattaaaaatctggacaaatccattgataataaagcactgtatgatacattttctgcttttggtaacatcctttcatgtaaggtggtttgtgatgaaaatggttccaagggctatggatttgtacactttgagacgcaggaagcagctgaaagagctattgaaaaaatgaatggaatgctcctaaatgatcgcaaagtatttgttggacgatttaagtctcgtaaagaacgagaagctgaacttggagctagggcaaaagaattcaccaatgtttacatcaagaattttggagaagacatggatgatgagcgccttaaggatctctttggcaagtttgggcctgccttaagtgtgaaagtaatgactgatgaaagtggaaaatccaaaggatttggatttgtaagctttgaaaggcatgaagatgcacagaaagctgtggatgagatgaacggaaaggagctcaatggaaaacaaatttatgttggtcgagctcagaaaaaggtggaacggcagacggaacttaagcgcaaatttgaacagatgaaacaagataggatcaccagataccagggtgttaatctttatgtgaaaaatcttgatgatggtattgatgatgaacgtctccggaaagagttttctccatttggtacaatcactagtgcaaaggttatgatggagggtggtcgcagcaaagggtttggttttgtatgtttctcctccccagaagaagccactaaagcagttacagaaatgaacggtagaattgtggccacaaagccattgtatgtagctttagctcagcgcaaagaagagcgccaggctcacctcactaaccagtatatgcagagaatggcaagtgtacgagctgttcccaaccctgtaatcaacccctaccagccagcacctccttcaggttacttcatggcagctatcccacagactcagaaccgtgctgcatactatcctcctagccaaattgctcaactaagaccaagtcctcgctggactgctcagggtgccagacctcatccattccaaaatatgcccggtgctatccgcccagctgctcctagaccaccatttagtactatgagaccagcttcttcacaggttccacgagtcatgtcaacacagcgtgttgctaacacatcaacacagacaatgggtccacgtcctgcagctgcagccgctgcagctactcctgctgtccgcaccgttccacagtataaatatgctgcaggagttcgcaatcctcagcaacatcttaatgcacagccacaagttacaatgcaacagcctgctgttcatgtacaaggtcaggaacctttgactgcttccatgttggcatctgcccctcctcaagagcaaaagcaaatgttgggtgaacggctgtttcctcttattcaagccatgcaccctactcttgctggtaaaatcactggcatgttgttggagattgataattcagaacttcttcatatgctcgagtctccagagtcactccgttctaaggttgatgaagctgtagctgtactacaagcccaccaagctaaagaggctgcccagaaagcagttaacagtgccaccggtgttccaactgtttaa

SEQ ID NO: 8 - protein, 636 aa, Homo sapiens
MNPSAPSYPMASLYVGDLHPDVTEAMLYEKFSPAGPILSIRVCRDMITRRSLGYAYVNFQQPADAERALDTMNFDVIKGKPVRIMWSQRDPSLRKSGVGNIFIKNLDKSIDNKALYDTFSAFGNILSCKVVCDENGSKGYGFVHFETQEAAERAIEKMNGMLLNDRKVFVGRFKSRKEREAELGARAKEFTNVYIKNFGEDMDDERLKDLFGKFGPALSVKVMTDESGKSKGFGFVSFERHEDAQKAVDEMNGKELNGKQIYVGRAQKKVERQTELKRKFEQMKQDRITRYQGVNLYVKNLDDGIDDERLRKEFSPFGTITSAKVMMEGGRSKGFGFVCFSSPEEATKAVTEMNGRIVATKPLYVALAQRKEERQAHLTNQYMQRMASVRAVPNPVINPYQPAPPSGYFMAAIPQTQNRAAYYPPSQIAQLRPSPRWTAQGARPHPFQNMPGAIRPAAPRPPFSTMRPASSQVPRVMSTQRVANTSTQTMGPRPAAAAAAATPAVRTVPQYKYAAGVRNPQQHLNAQPQVTMQQPAVHVQGQEPLTASMLASAPPQEQKQMLGERLFPLIQAMHPTLAGKITGMLLEIDNSELLHMLESPESLRSKVDEAVAVLQAHQAKEAAQKAVNSATGVPTV

SEQ ID NO: 9 - protein, 414 aa, Homo sapiens
MSEYIRVTEDENDEPIEIPSEDDGTVLLSTVTAQFPGACGLRYRNPVSQCMRGVRLVEGILHAPDAGWGNLVYVVNYPKDNKRKMDETDASSAVKVKRAVQKTSDLIVLGLPWKTTEQDLKEYFSTFGEVLMVQVKKDLKTGHSKGFGFVRFTEYETQVKVMSQRHMIDGRWCDCKLPNSKQSQDEPLRSRKVFVGRCTEDMTEDELREFFSQYGDVMDVFIPKPFRAFAFVTFADDQIAQSLCGEDLIIKGISVHISNAEPKHNSNRQLERSGRFGGNPGGFGNQGGFGNSRGGGAGLGNNQGSNMGGGMNFGAFSINPAMMAAAQAALQSSWGMMGMLASQQNQSGPSGNNQNQGNMQREPNQAFGSGNNSYSGSNSGAAIGWGSASNAGSGSGFNGGFGSSMDSKSSGWGM

SEQ ID NO: 10 - DNA, 3142 bp, Homo sapiens
ctctctccgccccgggtcgctgccgcctccgccgctttcgggcttcgcagcctgaggaaaaaaagagaaaaagataaaaaaaatctgaaaacgcttcaaaatcctgaaaaaaaaaaaggaaaagaaaaaacgaatcctcggagaacccgcggggaagtcactttcgtacgcttccggcctgccccgcgcccgccgccgcagcgcttggcgtccgtcggtctccgtccgtcggtccgggggtgagccgcccgcccggcccgccgtgccctccccccgctcgggccccgagccccgcgccccgcgcctgccccggcgcaccacgtgtccgtgctgcccttcgccgcccgcccggggctcgccgagtcggcgcccacaaagatttggtttccctctgccccggcggttgtaatcttaaaccgccggagcccgaggcctatatttatagagaaacgcgtgtccccgaggccgccgtgggcagcgtccggtcgcctcttaaaggatttttacccttcggaaggggattccccgtttaatttttttcctactttgattttttgaaatttggagcttcgcaccaggaccgcggagaagtgcaaagtcgcggggagggccgtattgtgcggagagccttttgtctgcggtgctgcggccgtgggagccggcccccgcctcccgtttccgtcccgtctccaagcccgccgactccagctcgtcctcgccgcgccggtgccacctgtgagccgcggcgcgggcccgggctccgaaggcgcccctttgtcctgcggcgggcccgataagaagtcctcctggcggggctcggggtggtggggggcggggagatgaacgctgcggccagcagctaccccatggcctccctgtacgtgggcgacctgcattcggacgtcaccgaggccatgctgtacgaaaagttcagccccgcggggcctgtgctgtccatccgggtctgccgcgatatgatcacccgccgctccctgggctatgcctacgtcaacttccagcagccggccgacgctgagcgggctttggacaccatgaactttgatgtgattaagggaaagccaatccgcatcatgtggtctcagagggatccctctttgagaaaatctggtgtgggaaacgtcttcatcaagaacctggacaaatctatagataacaaggcactttatgatactttttctgcttttggaaacatactgtcctgcaaggtggtgtgtgatgagaacggctctaagggttatgcctttgtccacttcgagacccaagaggctgccgacaaggccatcgagaagatgaatggcatgctcctcaatgaccgcaaagtatttgtgggcagattcaagtctcgcaaagagcgggaagctgagcttggagccaaagccaaggaattcaccaatgtttatatcaaaaactttggggaagaggtggatgatgagagtctgaaagagctattcagtcagtttggtaagaccctaagtgtcaaggtgatgagagatcccaatgggaaatccaaaggctttggctttgtgagttacgaaaaacacgaggatgccaataaggctgtggaagagatgaatggaaaagaaataagtggtaaaatcatatttgtaggccgtgcacaaaagaaagtagaacggcaggcagagttaaaacggaaatttgaacagttgaaacaggagagaattagtcgatatcagggggtgaatctctacattaagaacttggatgacactattgatgatgagaaattaaggaaagaattttctccttttggatcaattaccagtgctaaggtaatgctggaggatggaagaagcaaagggtttggcttcgtctgcttctcatctcctgaagaagcaaccaaagcagtcactgagatgaatggacgcattgtgggctccaagccactatatgttgccctggcccagaggaaggaagagagaaaggctcacctgaccaaccagtatatgcaacgagtggctggaatgagagcacttcctgccaatgccatcttaaatcagttccagcctgcagcgggtggctactttgtgccagcagtcccacaggctcagggaaggcctccatattatacacctaaccagttagcacagatgaggcctaatccacgctggcagcaaggtgggagacctcaaggcttccaaggaatgccaagtgctatacgccagtctgggcctcgtccaactcttcgccatctggctccaactggtaatgctccggcctctcgtggcctccctactaccactcagagagtcgggtctgagtgcccggaccgcttggctatggactttggtggggctggtgccgcccagcaagggctgactgacagctgccagtctggaggcgttcccacagctgtgcagaacttagcgccacgcgctgctgttgctgctgctgctccccgggctgttgccccctacaaatacgcctccagtgtccgcagccctcatcctgccatacagcctctgcaggcaccccagcctgcggtccatgtgcaggggcaggagccactgactgcctccatgctggctgcagcacccccccaggaacagaagcagatgctgggagaacgcttgttcccactcatccaaacaatgcattcaaatctggctgggaagatcacgggaatgctgctggagatagacaactctgagctgctgcacatgttagagtcccccgagtctctccgctccaaggtggatgaagctgtagcagttctacaggctcatcatgccaagaaagaagctgcccagaaggtgggcgctgttgctgctgctacctcttagacaaggaaaaaccgattcaaaagccaaataaccccttatggaattcaactcaaggtttgaagacttcctagcttgtcctatggacctcaacaccaaggattacaaattgcaaatttaataggtcattttgtatcaaaaggtcaattatgaagcacctagaatttttcaattatacgaatatgttctttgggttctgctgtggcccagacagtgttaactttttttttattgtgggttttgattttttcccccagaaattggttttatttgatgtacccaagtcttacgtttcccaataaagaaaaaaaatctccataaaa

SEQ ID NO: 11 - DNA, 5373 bp, Artificial
  synthetic nucleic acid
tagctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagccatgctctaggtaccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattggatccactcgagtggagctcgcgactagtcgattcgaattcgatatcaagcttatcgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgataccgtcgactcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctcgactagagcatggctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagagctttttgcaaaagcctaggcctccaaaaaagcctcctcactacttctggaatagctcagaggccgaggcggcctcggcctctgcataaataaaaaaaattagtcagccatggggcggagaatgggcggaactgggcggagttaggggcgggatgggcggagttaggggcgggactatggttgctgactaattgagatgcatgctttgcatacttctgcctgctggggagcctggggactttccacacctggttgctgactaattgagatgcatgctttgcatacttctgcctgctggggagcctggggactttccacaccctaactgacacacattccacagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccattcgacgctctcccttatgcgactcctgcattaggaagcagcccagtagtaggttgaggccgttgagcaccgccgccgcaaggaatggtgcatgcaaggagatggcgcccaacagtcccccggccacggggcctgccaccatacccacgccgaaacaagcgctcatgagcccgaagtggcgagcccgatcttccccatcggtgatgtcggcgatataggcgccagcaaccgcacctgtggcgccggtgatgccggccacgatgcgtccggcgtagaggatctggctagcgatgaccctgctgattggttcgctgaccatttccgggtgcgggacggcgttaccagaaactcagaaggttcgtccaaccaaaccgactctgacggcagtttacgagagagatgatagggtctgcttcagtaagccagatgctacacaattaggcttgtacatattgtcgttagaacgcggctacaattaatacataaccttatgtatcatacacatacgatttaggtgacactatagaatacacggaattaattc